SEQUENCE LISTING (21 sequences)

SEQ ID NO: 1 - protein, 276 aa, Thalassiosira pseudonana
MKRFTPRPGEKEEIAFEKAPMPAAVAHFTSEAAEKKAVIVEKAPMPVATQYFHDLDKEEKTSTVQSANYSQPPTDYELSPEEAARRKAMPVATRTFTPYDKRKCEEAYEKAPMPVATKYFTEEAAAKKAALAAEKPPMPVATQYFHDLEQDKKSSSSSDGYIDYSLPPAHEISEEEAARREAMPHAVRHFTPYDKQESEKTYEKTPVPIAVQHFTPPIVPKEDASMVDRTKMPIAVQKFTPEYQPKEKVEYKLDPMPVAMEYFTTGKGAVKANHSV

SEQ ID NO: 2 - DNA, 831 bp, Thalassiosira pseudonana
atgaagaggttcactcctaggcctggagagaaggaggagattgcgttcgaaaaggctcctatgcctgcagcggtagctcactttaccagtgaagcagccgaaaagaaggctgtcattgtcgaaaaggctccaatgccagtagcgacgcagtacttccacgatctcgacaaggaggaaaagacatcaactgttcagtctgccaactactcccagcctcctactgattacgagttgtctcccgaggaagctgccaggaggaaagcaatgcccgttgcgaccagaacattcaccccttatgacaagcgcaaatgtgaagaggcgtatgaaaaggctcccatgccggttgcaaccaagtacttcactgaggaagcagccgcaaagaaggctgctttggctgctgaaaagcctcctatgcctgttgcaactcaatacttccacgaccttgaacaggataagaagtcgtcgtcgtcgtcggatggttacattgactactcccttcctcctgcccacgagatctccgaggaggaggcagccaggagggaagccatgccccacgcggtccgtcactttacgccatacgataagcaggagtctgagaaaacgtatgaaaagactcctgttccaattgcagtccagcacttcacccctcccatcgttcctaaggaagacgcatccatggtggacaggacaaagatgccaattgctgtgcaaaagttcacacctgagtatcagccaaaggaaaaggttgagtacaagctagatccaatgcccgttgcaatggagtacttcactactggcaaaggagcagtgaaggcgaaccatagtgtctga

SEQ ID NO: 3 - DNA, 172 bp, Thalassiosira weissfloggi
gcccttcctccgggctgcctcctcctcggagatctcgtgggcaggaggaagggagtagtcaatgtaaccatccgacgacgacgacgacttcttatcctgttcaaggtcgtggaagtattgagttgcaacaggcataggaggcttttcagcagccaaagcagccttcaagggc

SEQ ID NO: 4 - DNA, 172 bp, Chaetoceros neogracilis
gcccttccttcgggctgcctcctcctcggagatctcgtgggcaggaggaagggagtagtcaatgtaaccatccgacgacgacgacgacttcttatcctgttcaaggtcgtggaagtattgagttgcaacaggcataggaggcttttcagcagccaaagcagccttcaagggc

SEQ ID NO: 5 - DNA, 169 bp, Thalassiosira antarctica
gcccttcctcgggctgcctcttcctcggagatctcgtgggcaggaggaagggagtagtcaatgtaaccatccgacgacgacgacgacttcttatcctgttcaaggtcgtggaagtattgagttgcaacaggcataggaggcttttcagcagccaaagcagcctaagggc

SEQ ID NO: 6 - DNA, 172 bp, Skeletonema costatum
gcccttcctcctgttagcctcttcctcggagatctcgtgggcaggaggaagggagtagtcaatgtaaccatccgacgacgacgacgacttcttatcctgttcaaggtcgtggaagtattgagttgcaacaggcataggaggcttttcagcagccaaagcagccttcaagggc

SEQ ID NO: 7 - DNA, 172 bp, Coscinodiscus wailesii
gcccttccttcgttttgcctcttcctcggagatctcgtgggcaggaggaagggagtactcaatgtaaccatccgacgacgacgacgacttcttatcctgttcaaggtcgtggaagtattgagttgcaacaggcataggaggcttttcagcagccaaagcagccttcaagggc

SEQ ID NO: 8 - DNA, 170 bp, Ditylum brightwelli
gcccttcctccgggttgctcttatcggagatctcgtgggcaggaggaagggagtagtcaatgtaaccatccgacgacgacgacgacttcttatcctgttcaaggtcgtggaagtattgagttgcaacaggcataggaggcttttcagcagccaaagcagccttcaagggc

SEQ ID NO: 9 - DNA, 166 bp, Thalassiosira oceanic
gcccttccttggggttgcagcttcagggctcaactcgtaatcagtgggtggctgagagtagtcgatagaaggagcggctgtcttcttcttctgatccatgtcgtaaaagtgtttcgccgcaacaggcatagggggcttttcaacaaccaaagcagccttcaagggc

SEQ ID NO: 10 - DNA, 166 bp, Thalassiosira weissfloggi
gcccttccttggggatggatgttcagggctcaactcgtaatcagtgggtggctgagagtagtcgatagaaggagcggctgtcttcttcttctgatccatgtcgtaaaagtgtttcgtcgcaacaggcataggaggcttttcagcagccaaagcagccttcaagggc

SEQ ID NO: 11 - protein, 53 aa, Thalassiosira antartica
AALAAEKPPMPVATQYFHDLEQDKKSSSSSDGYIDYSLPPAHEISEEEAARGR

SEQ ID NO: 12 - protein, 57 aa, Thalassiosira pseudonana
KAALAAEKPPMPVATQYFHDLEQDKKSSSSSDGYIDYSLPPAHEISEEEAARREAMP

SEQ ID NO: 13 - protein, 55 aa, Chaetoceros neogracilis
KAALAAEKPPMPVATQYFHDLEQDKKSSSSSDGYIDYSLPPAHEISEEEAARRKG

SEQ ID NO: 14 - protein, 55 aa, Thalassiosira weissfloggi; identical to SEQ ID NO: 13

SEQ ID NO: 15 - protein, 55 aa, Coscinodiscus wailesii
KAALAAEKPPMPVATQYFHDLEQDKKSSSSSDGYIEYSLPPAHEISEEEAKRRKG

SEQ ID NO: 16 - protein, 55 aa, Skeletonema costatum
KAALAAEKPPMPVATQYFHDLEQDKKSSSSSDGYIDYSLPPAHEISEEEANRRKG

SEQ ID NO: 17 - protein, 54 aa, Ditylum brightwelli
KAALAAEKPPMPVATQYFHDLEQDKKSSSSSDGYIDYSLPPAHEISDKSNPEEG

SEQ ID NO: 18 - protein, 53 aa, Thalassiosira weissfloggi
KAALAAEKPPMPVATKHFYDMDQKKKTAAPSIDYSQPPTDYELSPEHPSPRKG

SEQ ID NO: 19 - protein, 52 aa, Thalassiosira oceanica
KAALVVEKPPMPVAAKHFYDMDQKKKTAAPSIDYSQPPTDYELSPEAATPRK

SEQ ID NO: 20 - DNA, 5267 bp, Artificial
  RNAi Vector
agcttgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccgataccaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgccggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgagcggccgcgatatctggtcgcaacgggcattgctttcctcctggcagcttcctcgggagacaactcgtaatcagtaggaggctgggagtagttggcagactgaacagttgatgtcttttcctccttgtcgagatcgtggaagtactgcgtcgctactggcattggagccttttcgacaatgacagccttcttttcggctgcttcactggtaaagtgagctaccgctgcaggcataggagccttttcgaacgcaatctcctcaagcttgcggccgcatactggattggtgaatcaatgagccgtagcacaatggttacattcggctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattactttaagtctccttcgtctttggtgtaggcatgttggacacaacgaggtaaaacacaacacaaacaatgtgtccagcaaagtagtagctgctccagttctcccgggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgata

SEQ ID NO: 21 - DNA, 5684 bp, Artificial
  RNAi Vector
agcttgcgctttttccgagaactccccataagtcaacggctccaatcaagaatgtatccgacaacggcgagcatagcaacacgtccgtctttggagtagaatcatcatgttgtggatgaatacacagatgaatgacattaaaagcatgaacatgttagagagtaggaggtagagattgatatggtagcattgcgatgtttgtttttggtcagcatatgatgagtggataccaatatgatgaaagttgaatctcgcgtttgagctcagcggtacgttattgatcgaaagtagcctgatcaaaatccttggagagtacaagaggatcaaagaatccagtgggggcgataactccaagctcgttctcaaagaggcaatggaggtagaaactcatcccagttgagaagaagtgaaggcagtggcggtggcgaaagcagaggcaacgaggacagacttcctgtgggttgatgcaacgaatatttccagaaggagaagtttagagagttgaaccgctacctacaatgacaaagtatcgtatcgattttgatgttggttggttatgaattcaaactgtaagttggattgtgagaagatcagaagttgaacgaacacatctttccgatcattcacctccacactgcaacaacacggtacttcttccgcggcaggtctctgtcgccattctcttgtcctgttgttggctgtgagacgaggaaagcaacgacaagtttcacaaaagggagttcctttaacgagatatgttttttataaagagtcccaatagaaagacaaattgattcctccgtgcaaacgcgcaaataaacaccacgtccattatatccatatctttcagagtatccaacaagtgttgaaggacaggtagttgaagtaacgtatcttccccctcgactggatccatcaacaaggcgaacaaatccattcaacctctcataaattatctgatttaccaaaccgataccaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgccggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgagcggccgcaagcttgaggagattgcgttcgaaaaggctcctatgcctgcagcggtagctcactttaccagtgaagcagccgaaaagaaggctgtcattgtcgaaaaggctccaatgccagtagcgacgcagtacttccacgatctcgacaaggaggaaaagacatcaactgttcagtctgccaactactcccagcctcctactgattacgagttgtctcccgaggaagctgccaggaggaaagcaatgcccgttgcgaccagaacattcaccccttatgacaagcgcaaatgtgaagaggcgtatgaaaaggctcccatgccggttgcaaccaagtacttcactgaggaagcagccgcaaagaaggctgctttggctgctgaaaagcctcctatgcctgttgcaactcaatacttccacgaccttgatatctggtcgcaacgggcattgctttcctcctggcagcttcctcgggagacaactcgtaatcagtaggaggctgggagtagttggcagactgaacagttgatgtcttttcctccttgtcgagatcgtggaagtactgcgtcgctactggcattggagccttttcgacaatgacagccttcttttcggctgcttcactggtaaagtgagctaccgctgcaggcataggagccttttcgaacgcaatctcctcaagcttgcggccgcatactggattggtgaatcaatgagccgtagcacaatggttacattcggctagctaagatccaatggcaaggaccaagtgctggaacttgttttgctttagcagatcttagcgtgagaggtatttgtcctctgtcaggagtagatagtagatgttctttttaaactaaaatgctaactgttccgaattcctcatcgcagctaatccgtacatcaaaagacaaaatgctaggtatgtgtactacatctcctgttgctagataagacatatgataggaaacacaccatcaatagtcattgtagctttacttatactacgcatttgcactttcccctgagtggcagaggcgcattgagaaaatcgatctcaacatagtttatgtagcatcccctagatccattactttaagtctccttcgtctttggtgtaggcatgttggacacaacgaggtaaaacacaacacaaacaatgtgtccagcaaagtagtagctgctccagttctcccgggggatccactagttctagagcggccggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgata

(19) United States
(12) Patent Application Publication
(10) Pub. No.: US 2013/0333074 A1
(43) Pub. Date: Dec. 12, 2013

(54) ENHANCER OF CELL DIVISION

(71) Applicants: Thomas Mock, Norwich (GB); Rachel Elizabeth Hipkin, Norwich (GB)

(72) Inventors: Thomas Mock, Norwich (GB); Rachel Elizabeth Hipkin, Norwich (GB)

(73) Assignees: University of Washington through its Center for Commercialization, Seattle, WA (US); University of East Anglia, Norfolk (GB)

(21) Appl. No.: 13/984,470

(22) PCT Filed: Feb. 10, 2012
(86) PCT No.: PCT/GB2012/050305

§ 371 Date: Aug. 8, 2013

Related U.S. Application Data
(60) Provisional application No. 61/441,692, filed on Feb. 11, 2011.

(51) Int. Cl. C07K 14/405

(52) U.S. Cl. CPC C07K 14/405
USPC 800/298; 435/41; 435/69.1; 435/101; 435/134; 435/161; 435/468; 435/471; 435/183; 435/410; 435/419; 435/254.1; 435/254.11; 435/254.2; 435/255.1; 435/257.1; 435/257.2; 435/320.1; 530/370; 536/23.7

(57) ABSTRACT

The present invention relates to a polypeptide (BIG1) and variants thereof capable of enhancing the rate of cell-division of a microorganism or plant cell, as well as nucleic acid molecules encoding said polypeptides, vectors comprising said nucleic acid molecules and host cells transformed or transfected with said vectors and expressing said polypeptides. The BIG1 polypeptide which has been identified in the marine centric diatom Thalassiosira pseudonana, variants thereof and nucleic acids encoding these may be used in methods of enhancing the rate of cell-division of microorganisms, plant cells or plants which produce useful sub stances or exhibit useful properties, to increase the yield thereof.

This invention relates to a polypeptide (BIG1) and variants thereof capable of enhancing the rate of cell-division of a microorganism or plant cell, as well as nucleic acid molecules encoding said polypeptides, vectors comprising said nucleic acid molecules and host cells transformed or transfected with said vectors and expressing said polypeptides. The BIG1 polypeptide which has been identified in the marine centric diatom Thalassiosira pseudonana, variants thereof and nucleic acids encoding these may be used in methods of enhancing the rate of cell-division of microorganisms, plant cells or plants which produce useful substances or exhibit useful properties, to increase the yield thereof.

### INTRODUCTION

Diatoms are a major group of algae and one of the most common types of phytoplankton. Most diatoms are unicellular, although they can exist as colonies in the shapes of filaments or ribbons. A characteristic feature of diatom cells is that they are encased within a unique cell wall made of silica called a frustule. Marine diatoms exhibit a “bloom and bust” life cycle whereby they can very rapidly replicate when conditions are favourable (called a bloom) and can quickly dominate phytoplankton communities. This opportunistic growth is the reason why they contribute to about 25% of global carbon fixation. The mechanism that enables translation of favourable environmental conditions into a bloom has been hitherto unknown.

The present inventors have now identified a conserved DNA-associated protein and its encoding gene from the diatom Thalassiosira pseudonana which is a major regulator responsible for bloom formation in marine centric diatoms. The new gene, which was found to have no significant homology to any genes in the NCB1 dataset or uniprot dataset, has been named “bloom inducer gene” or BIG1.

In diatoms, culture in conditions of silicate limitation leads to cell cycle arrest at two points between G1 and S phase (just before DNA synthesis) and G2, prior to mitosis and cell division. The inventors had observed that BIG1 is upregulated in conditions of silicate limitation and is also upregulated during S phase (DNA synthesis). Thus, it was to be expected that BIG1 played a role in the cell cycle of marine diatoms. Further work, as described herein, has shown that over-expression, using a modified T. pseudonana expression cassette (Poulsen et al. 2006, Journal of Phycology 42, 1059-1065) of BIG1 in Thalassiosira pseudonana caused a distinct phenotype, characterised by fast recovery and growth after a period of nitrogen starvation, which lead to out competition of a wild-type culture. Comparative whole-genome expression profiling of the transgenic strain and wild type under simulated bloom conditions revealed that BIG1 regulates various transcription factors, DNA-methyltransferases, and RNA processing proteins among unknown diatom specific proteins. Many of these proteins regulated by BIG1 could be identified in a natural bloom of centric diatoms, confirming their significance for bloom formation.

Further, the inventors have confirmed that polypeptides having a common structural motif with BIG1 in a core region can be found in other centric diatoms. As shown herein, amino acids 128 to 184 of BIG1 share very high amino acid identity with these polypeptides from other diatoms.

In the light of these observations, the BIG1 gene and variants encoding a polypeptide with the function of BIG1 may be used to transfect or transform microorganisms, including yeast and fungi as well as plant cells to induce a rapid increase in cell-division (bloom) therein. Such an increase in yield would be very advantageous in the case of cells or plants which produce useful products such as, for example, biofuels or long-chain polyunsaturated fatty acids, as well as for general production of biomass and/or for agricultural crops. The invention is further described herein.

### DESCRIPTION OF THE INVENTION

In a first aspect the invention relates to a nucleic acid molecule encoding a polypeptide capable of enhancing the rate of cell-division of a microorganism or plant cell (activity of BIG1) wherein said polypeptide comprises an amino acid sequence having at least 50% amino acid sequence similarity with amino acids 128 to 184 of the amino acid sequence set forth in FIG. 1 or is a nucleic acid molecule complementary thereto. In one embodiment the nucleic acid molecule may encode a polypeptide having at least 50% amino acid sequence identity to the amino acid sequence of FIG. 1 or may be the complement thereof.

Preferably, the nucleic acid molecule encodes a polypeptide having at least 55%, at least 60%, at least 65%, at least 70%, at least 75%, at least 80%, at least 85%, at least 90% or at least 95% amino acid sequence similarity to the amino acids 128 to 184 of the sequence set forth in FIG. 1 or to the amino acid sequence of FIG. 1, most preferably across the entire length of the amino acid sequence set forth in FIG. 1.

In one embodiment the invention relates to a nucleic acid molecule wherein the encoded polypeptide comprises an amino acid sequence having at least 50% amino acid sequence identity to the amino acids 128 to 184 of the amino acid sequence set forth in FIG. 1 or is a nucleic acid molecule complementary thereto.

The percentage identity to amino acids 128 to 184 of FIG. 1 or the amino acid sequence set forth in FIG. 1 may be at least 55%, at least 60%, at least 65% at least 70%, at least 75%, at least 80%, at least 90% or at least 95% and is preferably across the entire length of the amino acid sequence of FIG. 1.

Preferably the nucleic acid molecule is one which encodes a polypeptide comprising the amino acid sequence set forth in FIG. 1.

In a second aspect the invention relates to a nucleic acid molecule encoding a polypeptide capable of enhancing the rate of cell-division of a microorganism or a plant cell wherein said nucleic acid molecule comprises a nucleotide sequence having at least 50% sequence identity to nucleotides 381 to 552 of the nucleotide sequence of FIG. 2 or the complement thereof.

Preferably, the nucleic acid molecule comprises a nucleic acid sequence having at least 50% identity to the nucleotide sequence of FIG. 2 or is the complement thereof.

The percentage identity of the nucleotide sequence to the nucleotides 381 to 552 of FIG. 2 or to the sequence set forth in FIG. 2 may be at least 55%, at least 60%, at least 65%, at least 70%, at least 75%, at least 80%, at least 85%, at least 90% or at least 95% and is preferably across the entire length of the nucleotide sequence of FIG. 2.

In one embodiment of the invention the nucleic acid molecule comprises the sequence of nucleotides set forth in FIG. 2.

In another embodiment the nucleic acid molecule which encodes a polypeptide capable of enhancing the rate of cell-division of a microorganism or plant cell is capable of hybridising under the medium conditions of stringency, preferably under conditions of high stringency to the complement of the nucleotide sequence set forth in FIG. 2.

The nucleic acids of the invention may be DNA or RNA and may be epigenetically modified, for example by means of cytosine methylation. Further, the nucleic acid molecule may include modified nucleotides.

In a third aspect the invention relates to a nucleic acid molecule capable of acting as a nucleic acid probe or primer and which comprises a fragment of the nucleotide sequence set forth in FIG. 2 or the complement thereof. Preferably said fragment is between 10 to 50 nucleotides in length or between 10 and 30 nucleotides in length.

In yet a further aspect there are provided nucleic acid vectors, preferably expression vectors comprising any one of the nucleic acid molecules discussed above, as well as host cells transformed or transfected with said vectors. The vectors may be constructed in a manner well-known to those skilled in the art.

Suitable host cells in which to express the nucleic acids of the invention and thereby enhance its cell-division rate are yeast, other fungal cells, algal cells or plant cells. For example the host cell may be a diatom. Preferably, the host cell is a photosynthetic cell. The transformation or transfection of such cells may be carried out in a manner well-known to those skilled in the art.

The invention thus also relates to a specific (isolated) strain of algae belonging to the Thalassiosiraceae family and in particular the genus Thalassiosira, more specifically a strain of Thalassiosira pseudonana (Thalassiosira pseudonana-1335-BIG1). The strain was deposited with the Culture Collection of Algae and Protozoa under the accession number CCAP 1085/23 and accepted on 7 Feb. 2011.

Transgenic plants comprising the nucleic acids of the invention and having an enhanced growth rate are also embodiments of the invention, as are transgenic or mutant algal cultures showing enhanced algal bloom as a result of enhanced or over-expression of the said nucleic acids.

The invention also relates to a vector comprising the antisense of the nucleic acid molecule described above, or a fragment thereof, under the control of a promoter. In a preferred embodiment, the fragment is nucleotides 33 to 282 of the nucleic acid molecule described above. Furthermore, the invention relates to a vector comprising an inverted repeat of the nucleic acid molecule described above, or a fragment thereof, under the control of a promoter. In a preferred embodiment, the fragment is nucleotides 33 to 446 of the nucleic acid molecule described above.

In a fourth aspect the invention relates to a polypeptide capable of enhancing the rate of cell-division of a microorganism or plant cell (activity of BIG1) wherein said polypeptide comprises an amino acid sequence having at least 50% amino acid similarity to amino acids 128 to 184 of FIG. 1 or at least 50% amino acid identity with amino acids 128 to 184 of the amino acid sequence set forth in FIG. 1.

Preferably the percent identity or percent similarity is at least 55%, at least 60%, at least 65%, at least 70%, at least 75%, at least 80%, at least 85%, at least 90% or at least 95% amino acid sequence similarity or identity to amino acids 128 to 184 of FIG. 1 or to the amino acid sequence set forth in FIG. 1, most preferably across the entire length of the amino acid sequence set forth in FIG. 1. In one embodiment the polypeptide of the invention (BIG1) comprises the amino acid sequence set forth in FIG. 1 or may comprise a polypeptide which differs from the sequence of FIG. 1 only by virtue of conservative amino acid changes.

The polypeptides of the invention may be formed into compositions for application to microorganisms and plant cells such as those recited herein to enhance the rate of cell-division thereof, for example for inducing “bloom”.

Alternatively, a method for enhancing the rate of cell-division of a microorganism or plant cell may be achieved by transforming or transfecting said microorganism or plant cell with a nucleic acid of the invention such that the encoded polypeptide is expressed therein. Preferably, the transfected or transformed cell is a yeast, a fungal cell, an algal cell or a plant cell. Such transformation or transfection may be carried out in any manner well-known to one skilled in the art.

The method of the invention can be used on microorganisms including algae, on plant cells or on a plant which have other genetic modifications, such as for example, cells which produce, biofuels, long-chain polyunsaturated fatty acids or other useful substances or activities. By enhancing the rate of cell-division or bloom, a much higher yield of the substance may be achieved. Indeed, there are many known industrial applications of algae such as those listed in Table 1 or Table 2 for which application of the method of the invention would be beneficial.

TABLE 1

Amino acids
Animal feed
Antibiotics
Antibodies
Catalysis
Chemical and biological sensing and
diagnosis
Computer chips
Cosmetics
Drug delivery systems
Energy storage including as capacitors
Enzymes
Ethanol production
Fatty acids
Feed additives
Feed surrogates
Fluid fuel
Food supplements
Foodstuffs
Fuel Production
Health food
Hormones
Immune modulators
Industrial waste detoxification
Lipids
Light-emitting display and optical storage
Microelectronic devices
Nanofiltration
Nanotechnologies
Natural oils for biodiesel production
Nitrogen-fixing biofertilizer
Pharmaceutically active substances
Phytoremediation of heavy metals
contamination
Pigments
Polysaccharides
Proteins for methane production
Raw materials
Renewable energy
Synthetic substances
Therapeutic supplements
Unsaturated fatty acids (e.g.
eicosapentaenoic acid, docosahexaenoic
acid and other omega-3 fatty acids)
Vaccines
Vitamins

TABLE 2
Energy (Biomass, Biomethane, Biofuel, Bio-oil, Biodiesel, Biohydrogen
(directly produced by algae)
High-value added products from algae(Small molecules, Polymers,
Hydrocolloids, Ulvan, Pharmaceuticals and cosmetics, High value oils,
Colourants, Materials)
CO2 mitigation and sequestration (CO2 mitigation, Carbon sequestration,
Carbon trading)
Waste water treatment (Removal of nutrients, Removal of organic
pollutants, Removal of heavy metals)

In addition, the nucleic acids and polypeptides of the invention may be used to increase the yield of the cells themselves, for example, for producing biomass or to increase the yield of an agricultural crop.

### DEFINITIONS

As used herein, sequence identity or percent identity is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:_{—}482-489 (1981). This algorithm may be extended to use with peptide or protein sequences using the scoring matrix created by Dayhoff, Atlas of Protein Sequences and Structure, M. O. Dayhoff ed., 5 Suppl. 3:_{—}353-358, National Biomedical Research Foundation, Washington, D.C., USA, and normalized by Gribskov, Nucl. Acids Res. 14_(6):_{—}6745-66763 (1986). The Genetics Computer Group (GCG) (Madison, Wis.) provides a computer program that automates this algorithm for both nucleic acid and peptide sequences in the “BestFit” utility application. The default parameters for this method are described in the Wisconsin Sequence Analysis Package Program Manual, Version 8 (1995) (available from GCG). Other equally suitable programs for calculating the percent identity or similarity between sequences are generally known in the art.
As used herein, “similarity” between two amino acid sequences is defined as the presence of a series of identical as well as conserved amino acid residues in both sequences. The higher the degree of similarity between two amino acid sequences, the higher the correspondence, sameness or equivalence of the two sequences. (“Identity between two amino acid sequences is defined as the presence of a series of exactly alike or invariant amino acid residues in both sequences)(see above).
As used herein, an example of medium stringency hybridization conditions includes hybridization in 4×sodium chloride/sodium citrate (SSC), at about 65-70° C. (or alternatively hybridization in 4×SSC plus 50% formamide at about 42-50° C.) followed by one or more washes in 1×SSC, at about 65-70° C. A preferred, non-limiting example of high stringency hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C.
As defined herein, conservative amino acid changes, refers to amino acid substitutions in which an amino acid residue is replaced with another amino acid residue of similar chemical structure and which has little or essentially no influence on the function, activity or other biological properties of the polypeptide.

Such conservative substitutions preferably are substitutions in which one amino acid within the groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn Glu and Gln; (c) polar, positively charged residues: His, Arg and Lys; (d) large aliphatic, nonpolar residues: Met, Leu He, Val and Cys; and (e) aromatic residues: Phe, Tyr and Trp.

The invention will now be demonstrated by virtue of the following non-limiting Figures and Examples.

### DESCRIPTION OF THE FIGURES

FIG. 1 shows the amino acid sequence of the bloom inducing gene BIG1 from T. pseudonana (SEQ ID No: 1);

FIG. 2 shows the nucleotide sequence of a nucleic acid molecule encoding BIG1 from T. pseudonana (SEQ ID No: 2);

FIG. 3 shows both the nucleotide sequence (SEQ ID No: 2) and the amino acid sequence (SEQ ID No: 1) encoded thereby for BIG1 from T. pseudonana;

FIG. 4 is a nucleic acid alignment of the core region of BIG1 amplified in other centric dictoms; Ta-Thalassiosira antartica (SEQ ID No: 5), tw-Thalassiosira weissfloggi (SEQ ID Nos: 3 & 10), to-Thalassiosira oceanic (SEQ ID No: 9), Db-Ditylum brightwelli (SEQ ID No: 8), cw-Coscinodiscus wailesii (SEQ ID No: 7), sc-Skeletonema costatum (SEQ ID No: 6), cn-Chaetoceros neogracilis (SEQ ID No: 4);

FIG. 5 is an amino acid alignment of the core region of BIG1 amplified in centric diatom species: Highlighted section indicates predicted coiled region regions (Lupas et al., 1991). Boxed region identifies two isoforms of BIG1;

FIG. 6 shows fluorescent microscope images of BIG1 transformants of T. pseudonana which over-express BIG1. Light images, chlorophyll autofluorescence, GFP fluorescence, and Hoechst stained cells are presented from two different over-expression clones (#21 and #25) of BIG1 with GFP. Images were taken with a Wide-field, CCD camera;

FIG. 7 shows growth of BIG1 over-expression mutant (biological replications 3) and WT (biological replications 3) post 7 days in nitrate limited stationary growth. Boxes indicate the time point at which harvesting was carried out for microarray analysis of cells;

FIG. 8 shows the results of a competition experiment in which 25,000 cells/ml (3 biological replicates) of BIG1 over-expression mutant and WT were inoculated into nutrient replete media and the percentage of cells recorded on a flow cytometer. Total cell counts for the population was performed to monitor growth;

FIG. 9 shows analysis of those genes from microarrays that are differentially upregulated by the over-expression of BIG1 present in eukaryotic metatranscriptome datasets of algae from Equatorial Pacific, Pudget Sound (both Mock et al., in prep) and a metatranscriptome dataset of an iron enriched sub sample of a natural phytoplankton population in a carboy experiment from Ocean Station Papa(OSP; 50 oN and 145 oW) Pacific (Armbrust et al., in prep; data available at CAMERA (http://camera.calit2.net/index.shtm)); normalised read counts of those reads with more than 10⁻⁵ homology qualified as significant alignments; and

FIG. 10 shows Rosetta transformed with BIG1 in the Pet21 vector. Lanes from left to right, protein ladder, overnight induction with IPTG of Pet21 BIG1 no GFP, no induction of Pet21 BIG1, overnight induction with IPTG of Pet21 BIG1 GFP and no induction of Pet21 BIG1 GFP.

FIG. 11 shows Natural Log Cells/mL and Fv/Fm of three biological replicates of Wildtype and BIG1 1(21) in nutrient replete media post 80 uM silicate yield limitation for 8 days.

FIG. 12 shows a diagram of an RNAi knockdown vector.

FIG. 13 shows the nucleic acid sequence of the vector of FIG. 12 (SEQ ID No: 20).

FIG. 14 shows a diagram of a second RNAi knockdown vector.

FIG. 15 shows the nucleic acid sequence of the vector of FIG. 14 (SEQ ID No: 21).

FIG. 16 shows a Western blot image showing the comparison of the BIG1 protein content of clones A2 and A3 transformed with the inducible antisense vector on the nitrate reductase promotor. When cells were grown in NH₄-containing NEPCC (hence with the silencing turned off) the BIG1 protein content is higher than when cells were grown in NO₃ containing media (hence with the silencing turned off).

FIG. 17 shows cell counts of wild type T. Pseudonana and a clone with the BIG1 gene knocked down using the inverted repeat vector (FIGS. 14 & 15), plotted against time after innoculation of cells from nitrate limited media into replete NEPCC.

### EXAMPLES

### Example 1

### Nuclear Targeting

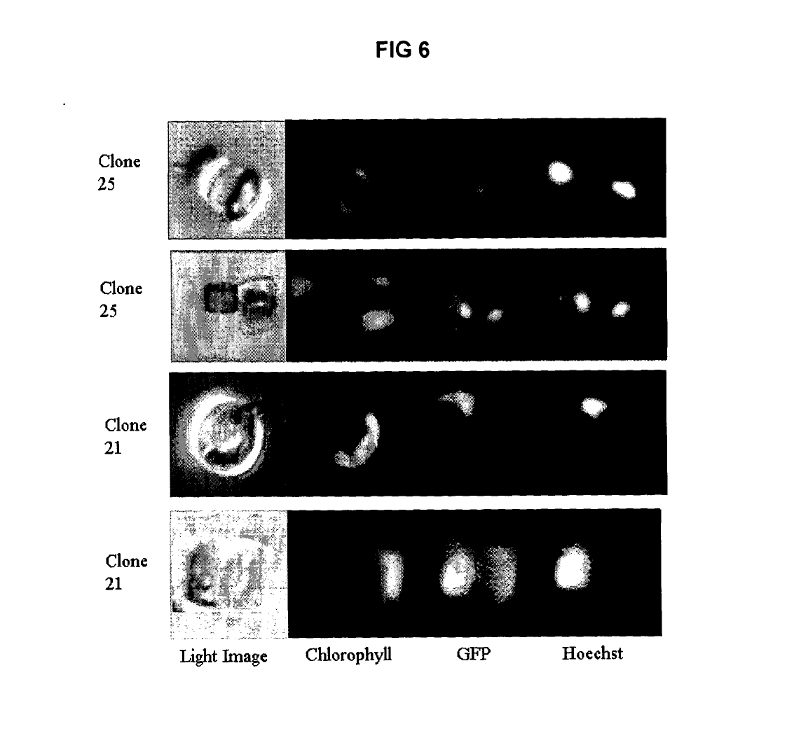

To investigate the role this gene could potentially play in regulation networks/transcription T. pseudonana was transformed with a BIG1 nitrate reductase-inducible over expression vector, tagged with green fluorescent protein (GFP). When DNA was extracted GFP was found to be bound to the DNA in vitro. The GFP signal has been shown to correspond to the nucleus as localised by the double stranded DNA stain Hoechst 33342 in vivo (see FIG. 6). Two clones were used to identify that this was not an artefact from the random integration of the GFP tagged BIG1 gene in T. pseudonana.

### Example 2

### Growth Experiments-Phenotype of Over-expression Mutant

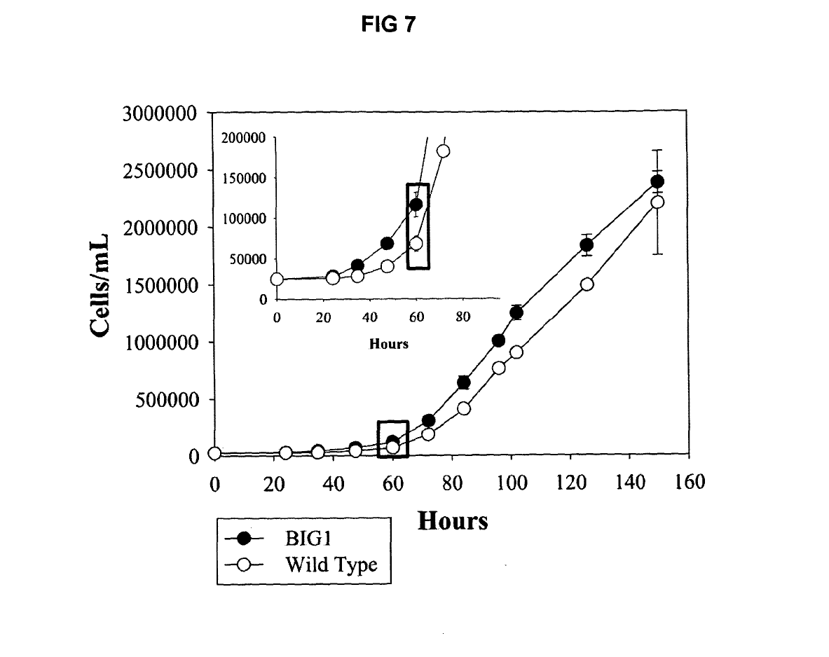

Growth experiments were carried out to obtain a phenotype for the over-expression of BIG1 in T. pseudonana. When mutants and wildtype (WT) are grown to limited states with a subsequent stationary phase (no growth) and then transferred into nutrient replete media the BIG1 over-expression cells are able to adjust to the nutrients and come out of a lag phase 24-48 hours before the WT cells. This phenotype was strongest when in a 100 uM nitrate concentration seawater with 7 days in stationary period then transferred to replete media (see FIG. 7).

### Example 3

### Competition Experiment

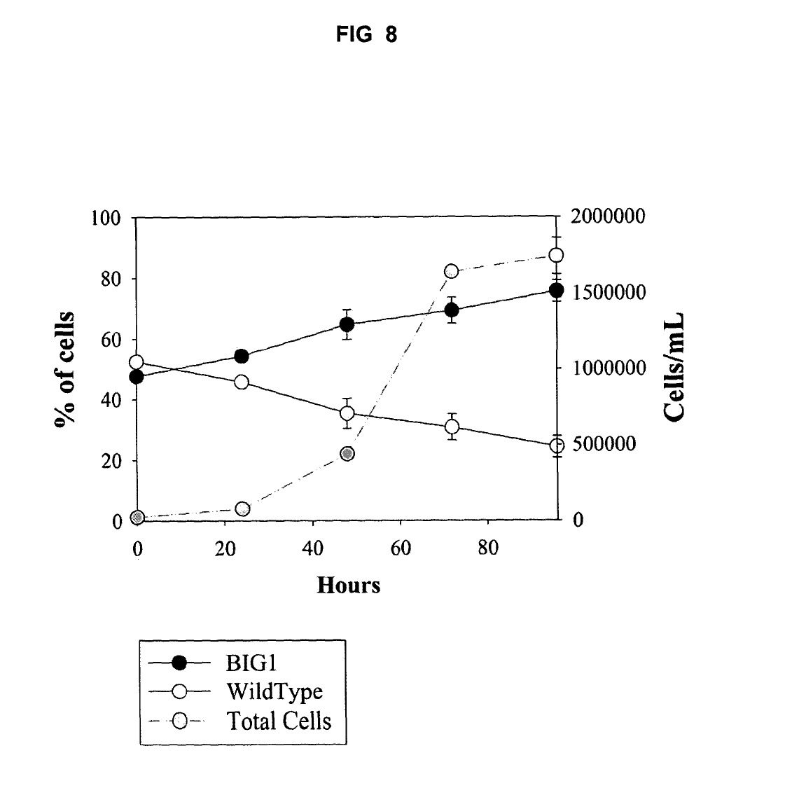

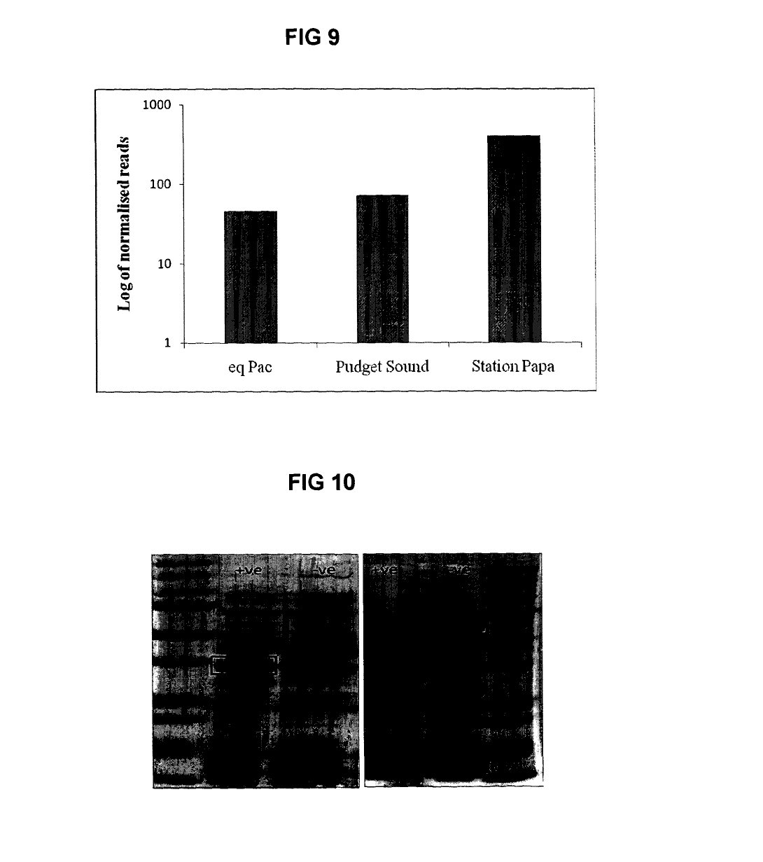

The competitive phenotype conferred by the over-expression of BIG1 was verified with a competition experiment (FIG. 8). The competition experiment was performed on a flow cytometer which distinguished with auto fluorescence and GFP fluorescence between the two populations of the WT and the BIG1 over-expression mutant. Both populations were gated to identify the percentage of BIG1 mutants and WT mutants in the same seawater. Both cell types were counted on a coulter counter initially so equal numbers of 25000 cells/ml of mutant and WT cells were added to the seawater post 7 days in a nitrate induced stationary period. The initial inoculums were verified on the flow cytometer where the ratios between WT and BIG1 gated population was 52/48%, respectively. Total cell counts of the mixed population were also performed to follow the growth of cells to stationary phase. After 96 hours after the co-inoculation of the two cell populations, when cells had reached the end of the growth period, the ratios between the WT and BIG1 had changed to 25/75%, respectively.

### Example 4

### BIG1 in Other Centric Diatoms

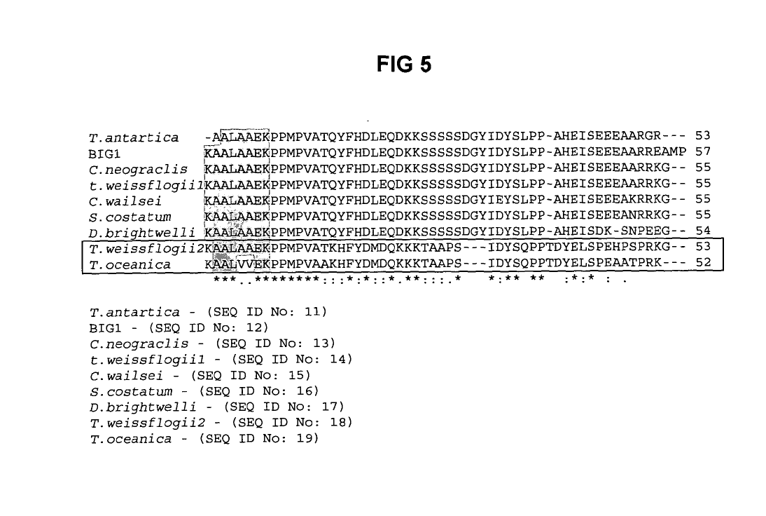

BIG1 is not present in P.tricornutum or F. cylindrus. To determine whether it had evolved in other centric diatoms clone libraries of other centric diatoms from the core region in the BIG1 gene flanked by repeats were prepared. BIG1 has been identified in 7 centric species (see FIG. 5). This clone library identified a different isoform of BIG1 (in T. oceanica and T. weissflogii2). T. weissfloggi was found to have both isoforms. The repeat region was chosen as it is predicted to contain a region with COILS, an alpha helices (Lupas et al., 1991, Science 252 (5010:1162-4).

The centric diatoms in which BIG1 homologues have been found come from different clades of centric diatoms (Damaste et al., 2004, Science 304 (584-587)

### Example 5

### Microarrays with BIG1 Over-expression Mutants and Wild Type

To analyse the effect of BIG1 on the whole gene expression of T. pseudonana, microarrays were carried out. The RNA samples used were at the point where BIG1 was more competitive in exponential phase (FIG. 7) and also from cells in day 7 of stationary phase following pre inoculation to nutrient replete media. An 8 by 16k microarray was carried out with 3 biological replicates for both cell types in exponential growth. Two extra samples of cells in nitrate limitation were also analysed.

The microarrays gave an insight to how BIG1 influences gene expression in T. pseudonana. There were 68 differentially upregulated genes and 36 downregulated genes in exponential growth, all p<0.05 with differential expression of more than log2 >1.0

Set forth below is a table focusing on the top 10 differentially up and down regulated genes in exponential growth in the BIG1 mutant (Table 3). Within the Top 10 only three have a known function, predicted by pfam/interpro p<10⁻⁵. All of these have a predicted function in cell signalling or transcription. It is interesting that in the top 10 there is one transcription factor and it is a myb transcription factor. This is relatively unexpected due to the expansion of the heat shock factors in T. pseudonana but not the Myb transcription factors (Montsant et al., 2007) (Plant Physiology, 10.1104/pp. 104.052829)). Within the top 10 there is also a calcium binding protein, likely regulating signalling. Also the presence of a cyclic nucleotide binding domain could represent signalling, since theses are recognised secondary messengers found in all kingdom of life (Beano & Brunton, 2002)(Nat Rev Mol Cell Biol. 2002 September; 3(9):710-8.).

Thus, in the downregulated dataset there is one gene potentially involved in down regulation of methylation. This dataset lead the inventors to carry out an analysis of the methylated state of the cells using an imprint methylation kit (Imprint® Methylated DNA Quantification, SigmaAldrich). BIG1 was found in exponentially growing cells to have a methylation of 15% of control DNA and WT was found to have 67% global methylation of the control DNA, control DNA was at 100%. The significance level was p=0.019 (N=3). The BIG1 over-expression mutant was found to be hypomethylated compared to the WT. This is extremely important as it indicates methylation patterns are important in growth of centric diatoms.

TABLE 3
Up and Down regulated genes in the Big1 over-expression mutant in
exponential growth relative to a WT culture. Log 2 change of
differential gene expression in BIG1 is supported by a p-value of <0.001.

| | Log 2 change in expression | |
|---|---|---|
| | relative to WT culture (+ | |
| | indicates upregulation; − down- | |
| Protein Id | regulation relative to WT culture) | Interpro id |
| 12185 | +2.87 | No Interpro ID |
| 260844 | +2.59 | IPR018248 |
| 4082 | +2.55 | No Interpro ID |
| 9775 | +2.39 | IPR019410; |
| | | IPR002761 |
| 32880 | +2.26 | IPR000595 |
| 11156 | +1.94 | No Interpro ID |
| 2250 | +1.90 | No Interpro ID |
| 10374 | +1.84 | No Interpro ID |
| 6097 | +1.77 | No Interpro ID |
| 7647 | +1.76 | IPR014778 |
| 8776 | −2.90 | IPR019410 |
| 21433 | −2.46 | No Interpro ID |
| 24954 | −2.02 | IPR000910 |
| 3054 | −1.99 | IPR003495; |
| | | IPR011629 |
| 8720 | −1.72 | No Interpro ID |
| TP_1_003255 | −1.70 | No Interpro ID |
| TP_1_000642 | −1.68 | No Interpro ID |
| 10194 | −1.63 | No Interpro ID |
| TP_1_002914 | −1.48 | No Interpro ID |
| TP_1_002071 | −1.33 | No Interpro ID |

As growth regulators were identified in the dataset, a further analysis was carried out to identify whether any of the differentially regulated genes were present in pennate diatoms. This analysis included the stationary dataset of gene expression. The analysis found that the most downregulated gene, the methyltransferase, is in F. cylindrus. Furthermore, 73 of the 309 genes were found in F. cylindrus and 23 in P. tricornutum (9 of these share with each other).

Following the finding of some of the differentially regulated genes in pennate diatoms, it was investigated whether any of these genes were being expressed in the environment, and thus are globally important. Eukaryotic metatranscriptomes were utilised from different environments and examined using bioinformatics. The datasets analysed were from the Equatorial Pacific, an oligotrophic environment, Pudget Sound, a coastal nutrient rich centric diatom bloom and an Iron induced pennate diatom bloom at Station Papa, Pacific.

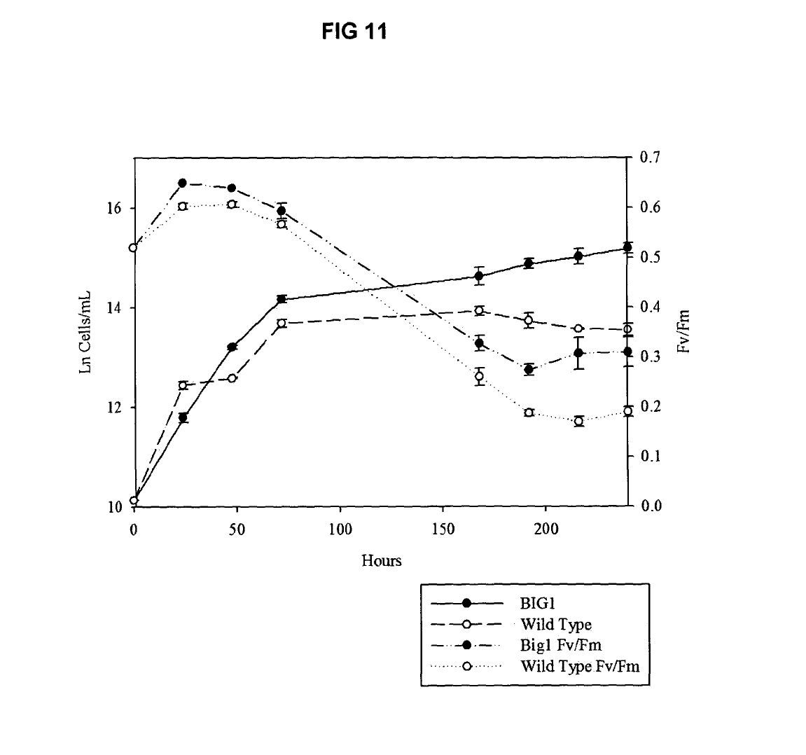

The number of normalised reads and transcripts from these datasets increased with nutrient availability, Equatorial Pacific, Pudget Sound and Station P (FIG. 11). The most reads and transcripts came from the bloom of pennate diatoms.

Thus, although BIG1 is not present in pennate diatoms they could have evolved similar networks to T. pseudonana for rapid growth with a pulse of a limiting nutrient. Moreover analysis of genes differentially regulated in the two genomes of pennate diatoms P. tricornutum and F. Cylindrus indicates many shared genes, between the two species and the pennate diatom bloom. The pennate bloom was dominated by Pseudo-nitzschia granii which is evolutionarily closer to F. Cylindrus than P. tricornutum and hence they have more shared genes between them.

### Example 6

### Expression in E.coli

BIG1 has been cloned in E.coli. It was cloned into Rosetta using in Pet 21 (no HIS tags), and inducible expression has been confirmed (FIG. 10).

### Example 7

### Nutrient Replete Growth Post Silicate Limitation

To identify phenotypes of BIG1 cells compared to WildType post a silicate induced stationary phase cells were first grown in reduced silicate seawater to 80 μM, compared to normally being 105 μM and doubling all other nutrients (other than vitamins which were kept at 1× concentration). Once stationary phase was reached no nutrients were added and cells were held in stationary phase for 8 days. After 8 days cells were inoculated at 25,000 cells/mL into nutrient replete seawater and cells/mL and Fv/Fm were recorded daily (FIG. 11).

### Specific Growth Rate

For the first 72 hours in nutrient replete media both BIG1 and WildType cells grow exponentially. The specific growth rate of each type of cell is shown in table 4. BIG1 cells were found to be growing significantly faster using a paired T-Testp<0.01, n=3 over the first 72 hours.

TABLE 4
Specific Growth Rate of BIG1 transgenic cell
line#21 and WildType T. pseudonana.

| | Mean Specific Growth Rate | Std Dev |
|---|---|---|
| BIG1 | 0.018778 | 0.00022 |
| WildType | 0.015033 | 0.00029 |

### Cell Yield

BIG1 cells also had significantly higher final cell yields than WildType using a paired T-Testp<0.01, n=3.

TABLE 5
At 216 hours post inoculation into nutrient replete
media average cells/mL and standard deviation n = 3

| | Cells/mL | Std Dev |
|---|---|---|
| BIG1 | 3594333.33 | 602113.22 |
| WildType | 1116000.00 | 150263.10 |

### Photosynthetic Efficiency

BIG1 cells were also found to have a significantly better photosynthetic efficiency using a paired T-Testp<0.01, n=3 with Fv/Fm at 216 hours being recorded as 0.35 higher than WildType cells, Table 6.

TABLE 6
At 216 hours post inoculation into nutrient replete
media average Fv/Fm and standard deviation n = 3

| | Fv/Fm | Std Dev |
|---|---|---|
| BIG1 | 0.49 | 0.04 |
| WildType | 0.14 | 0.05 |

### Example 8

### RNAi Knockdown Experiments

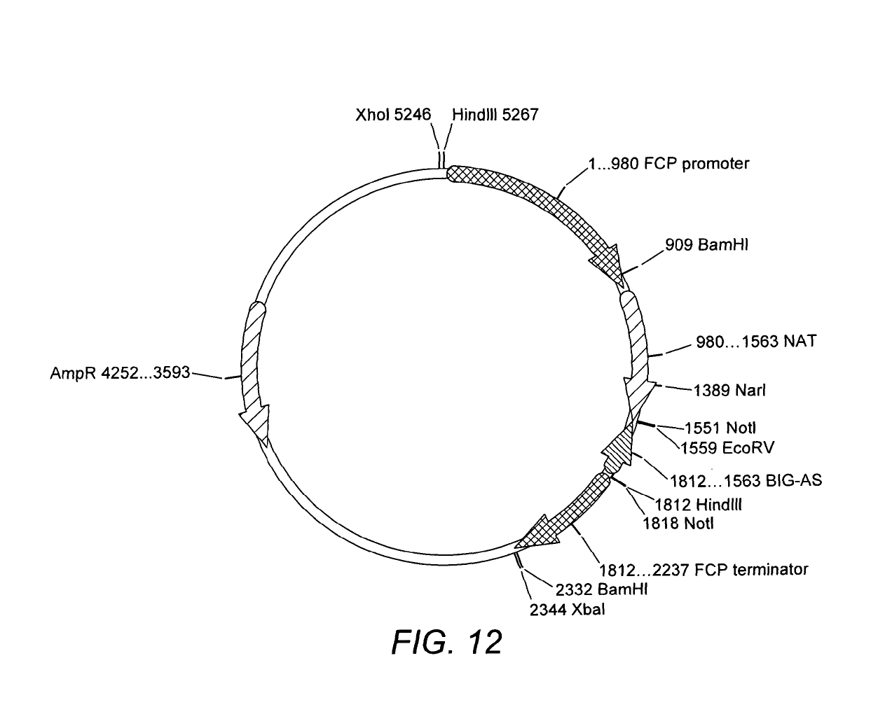

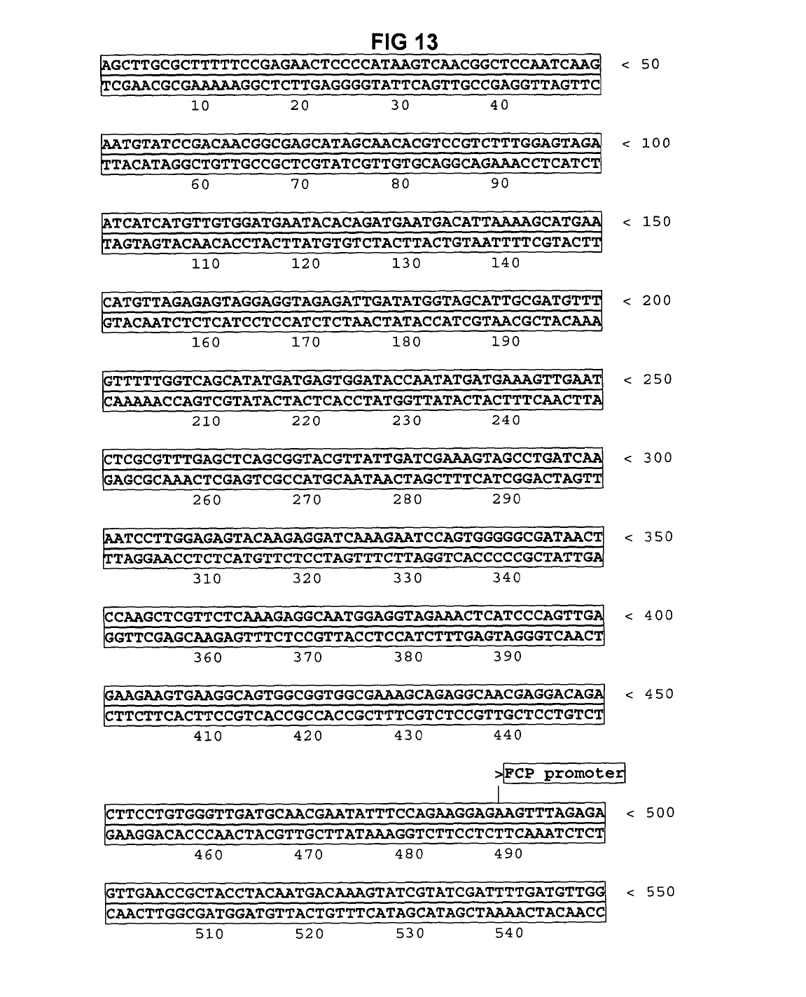

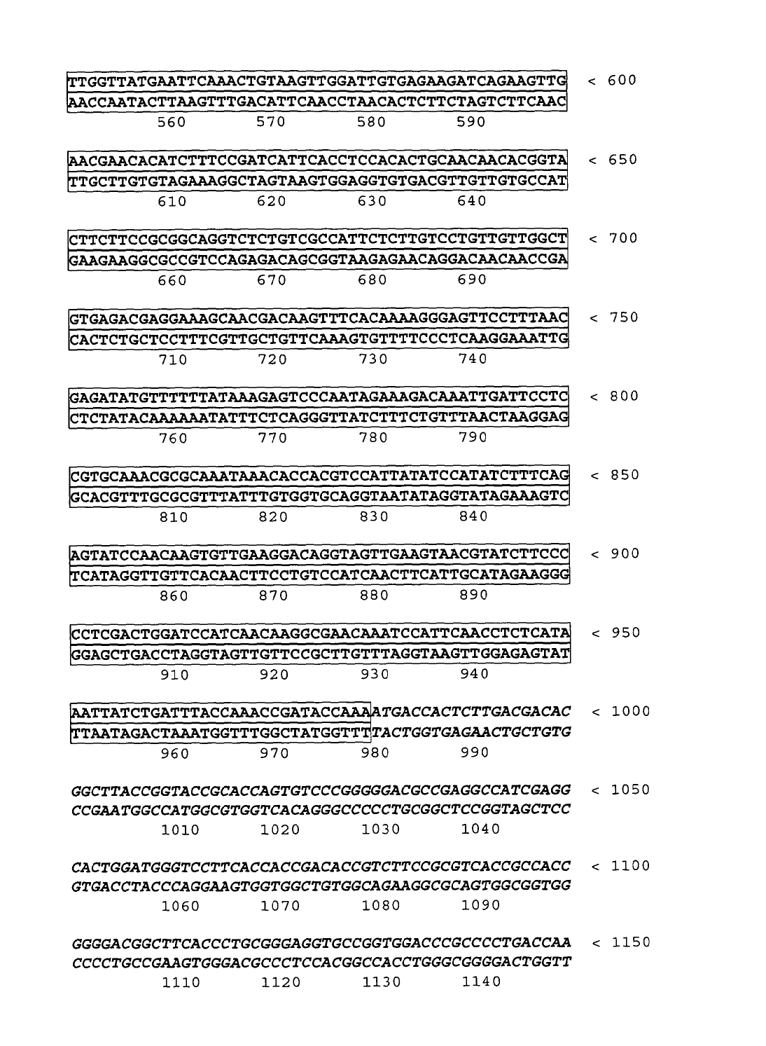

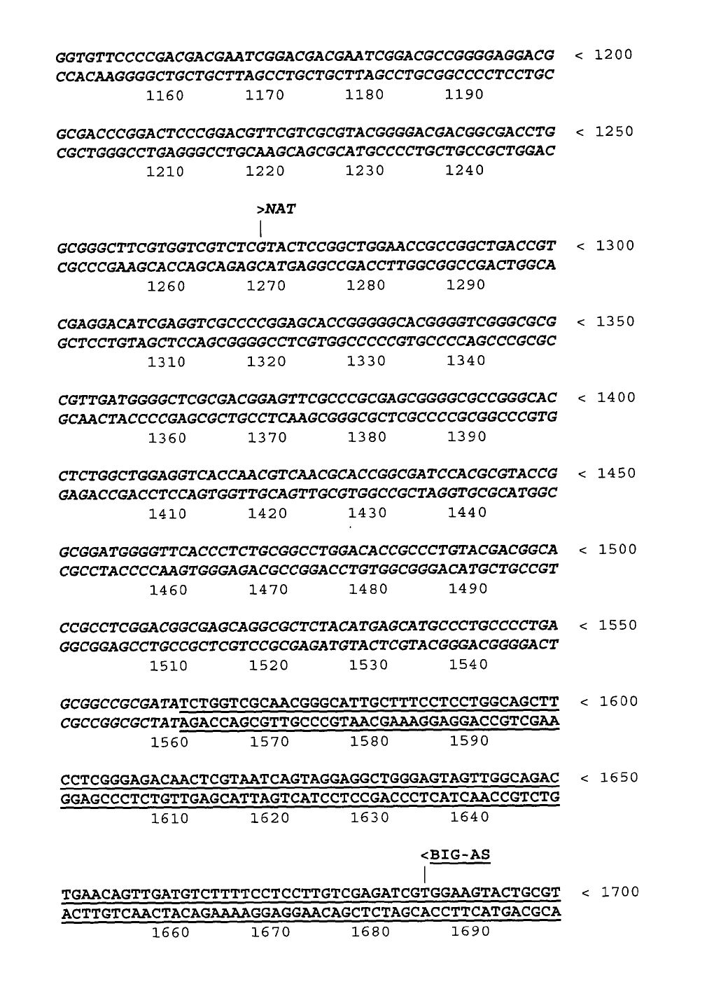

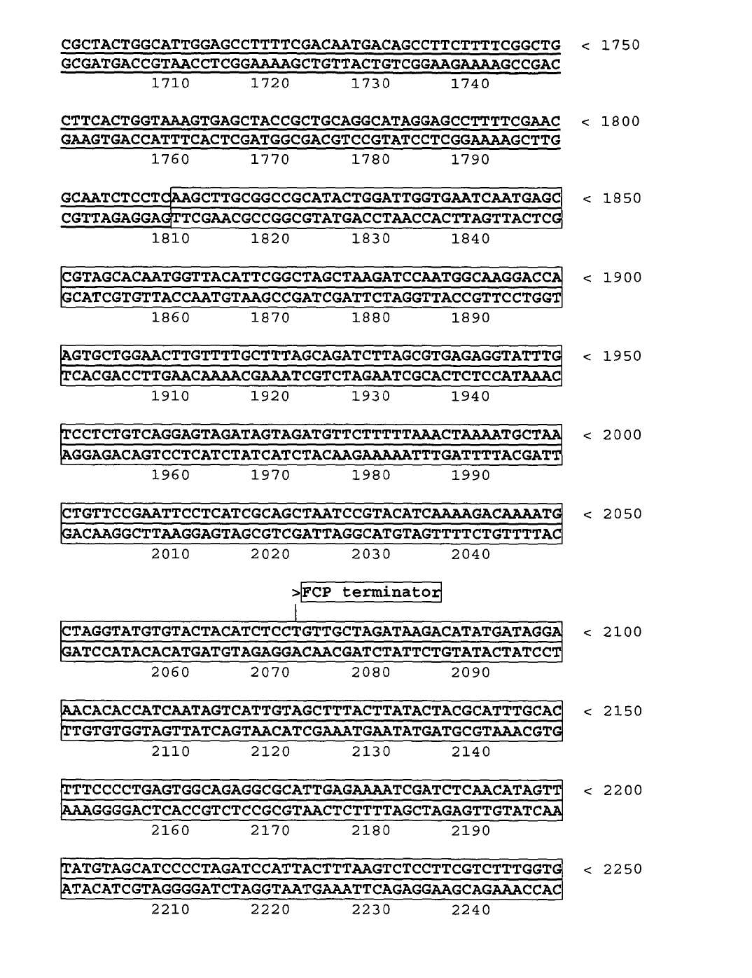

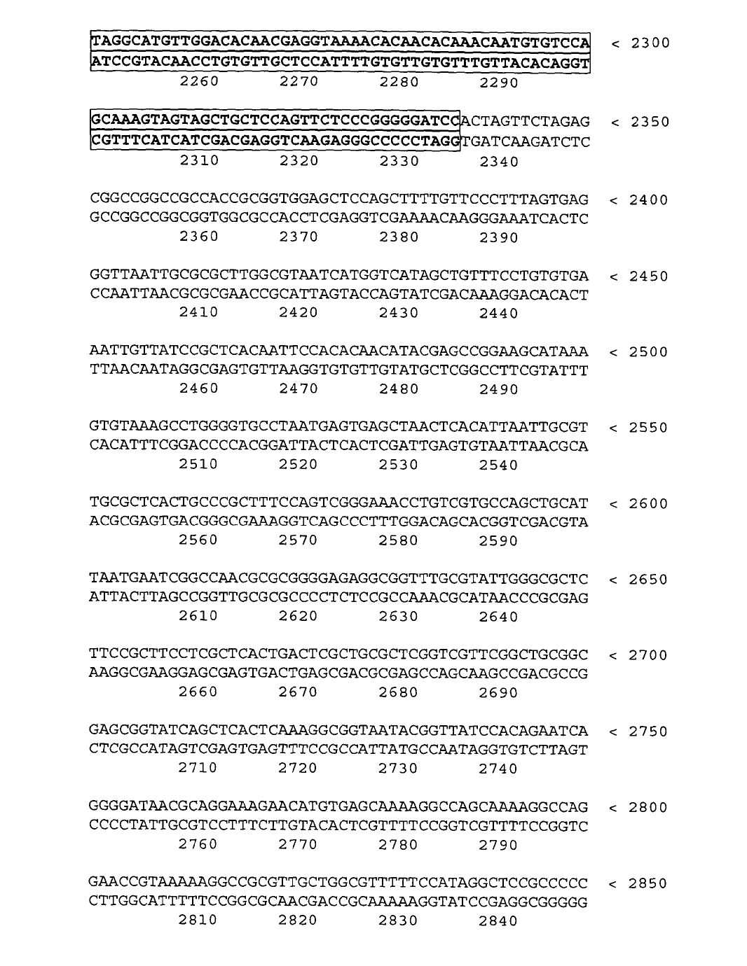

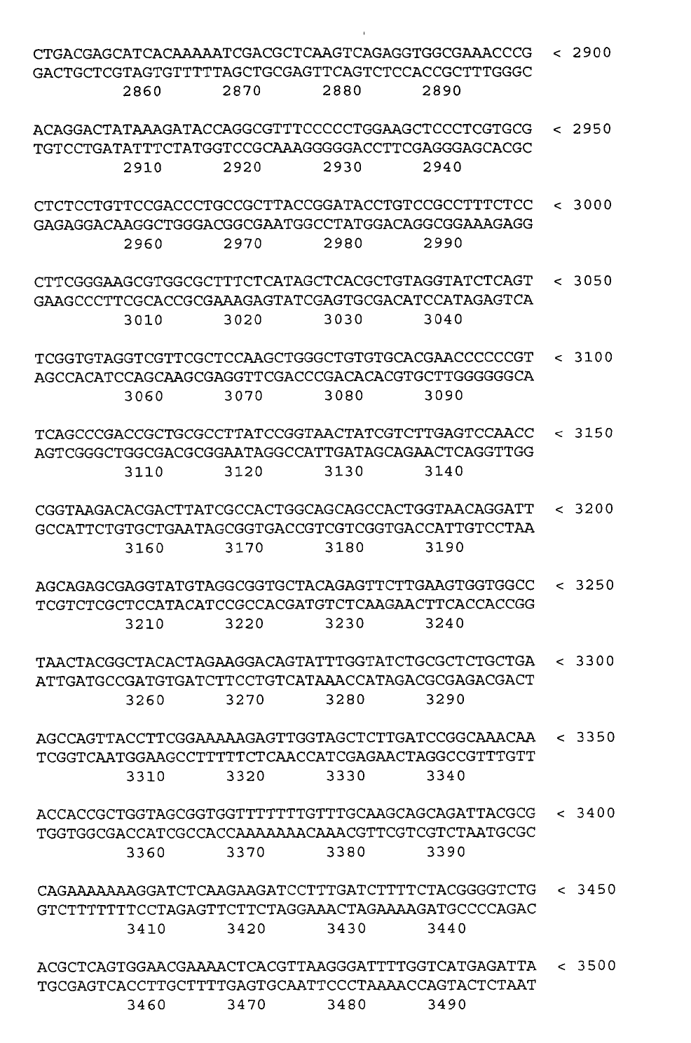

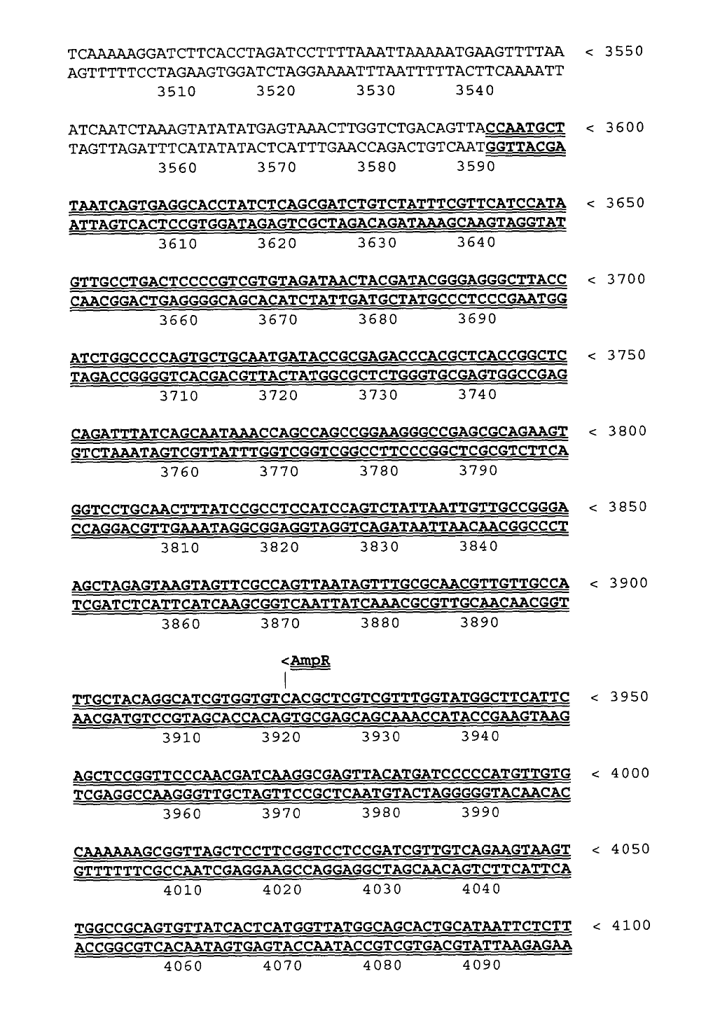

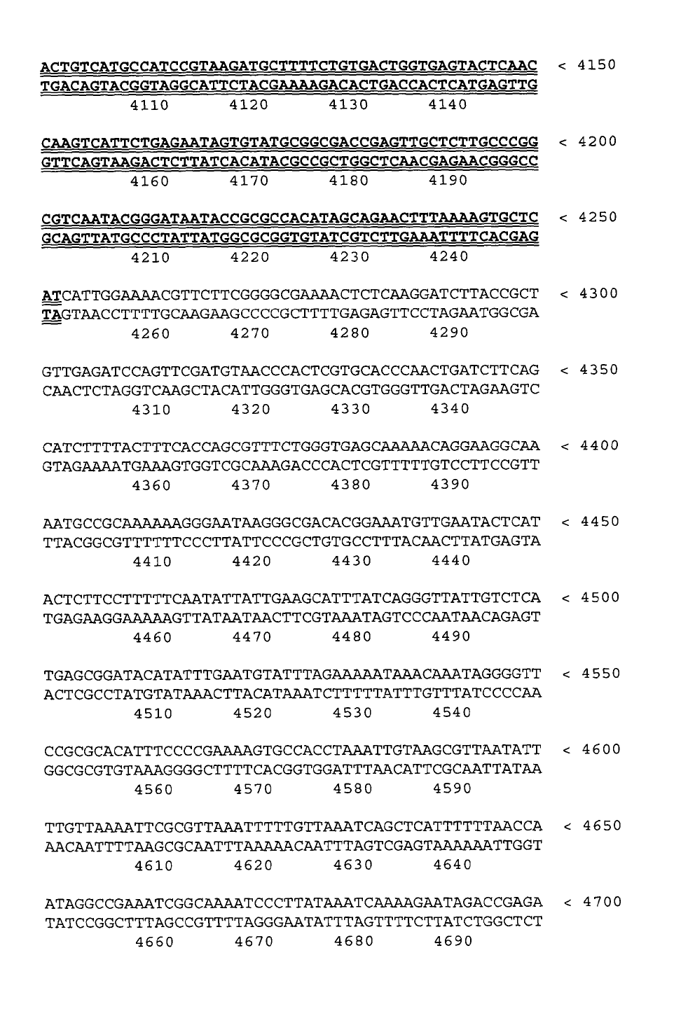

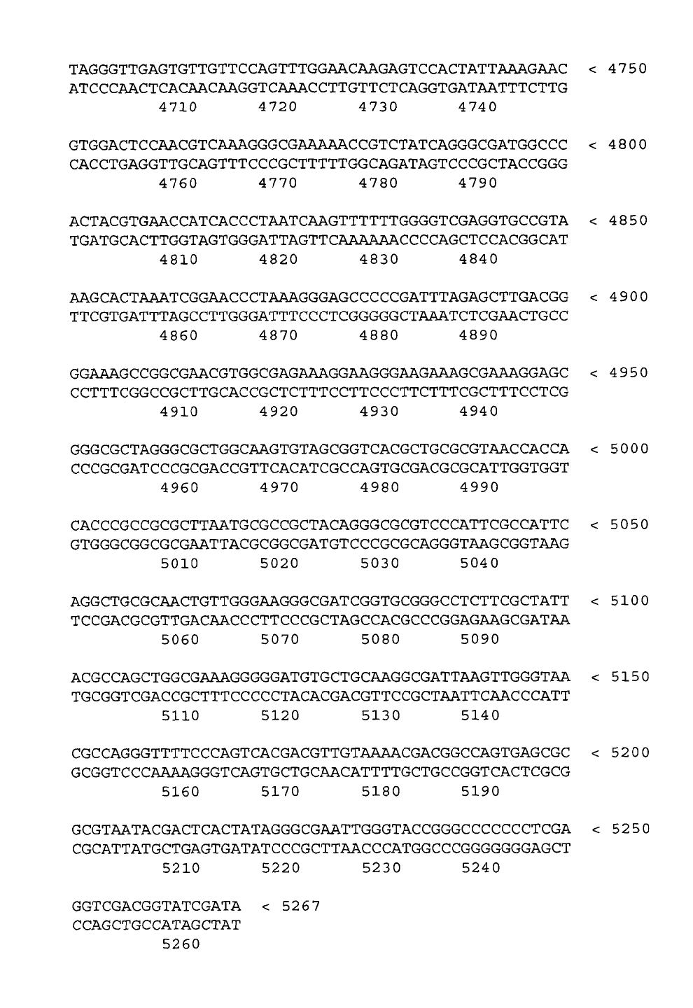

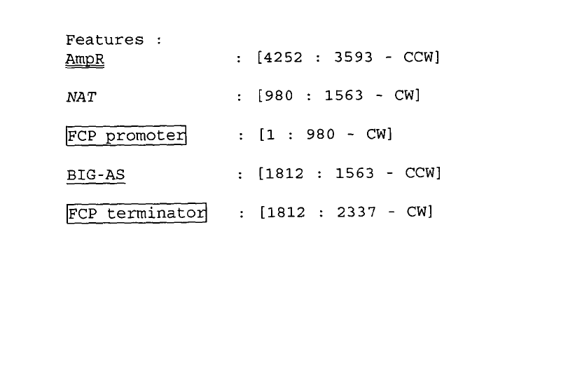

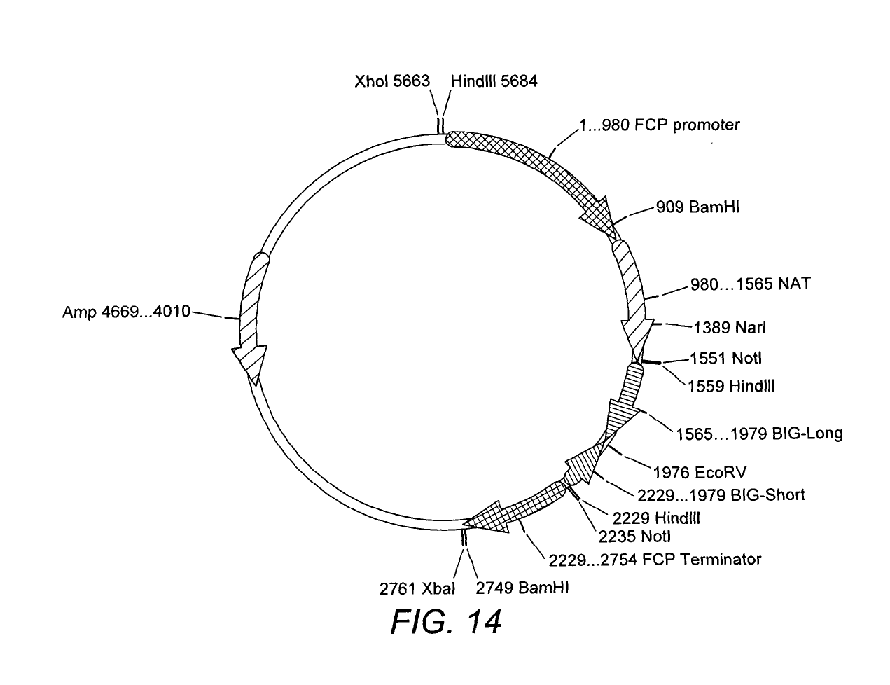

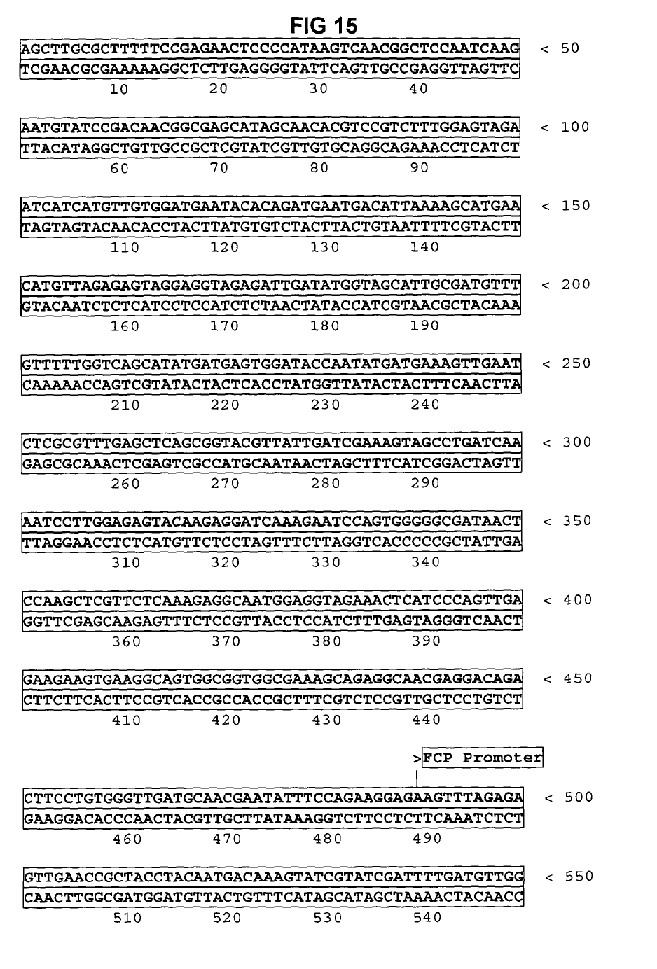

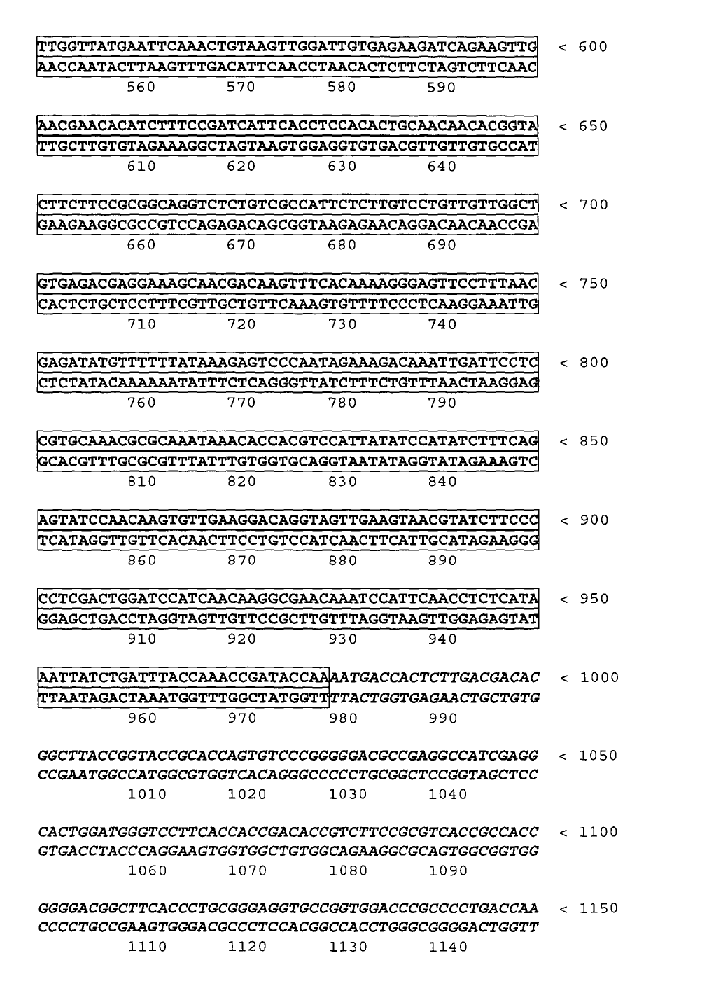

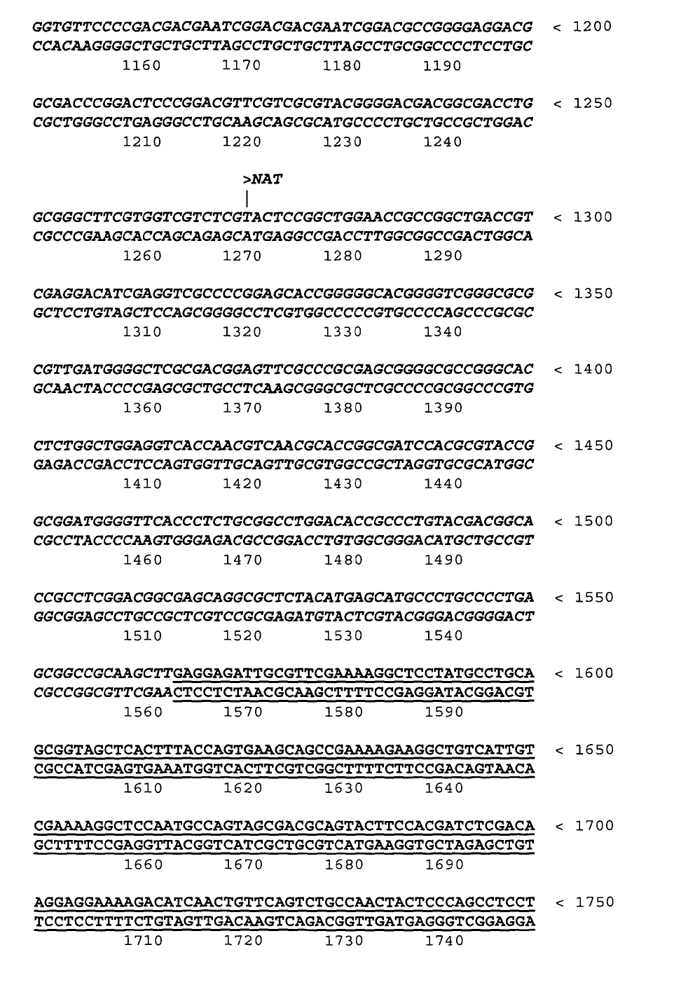

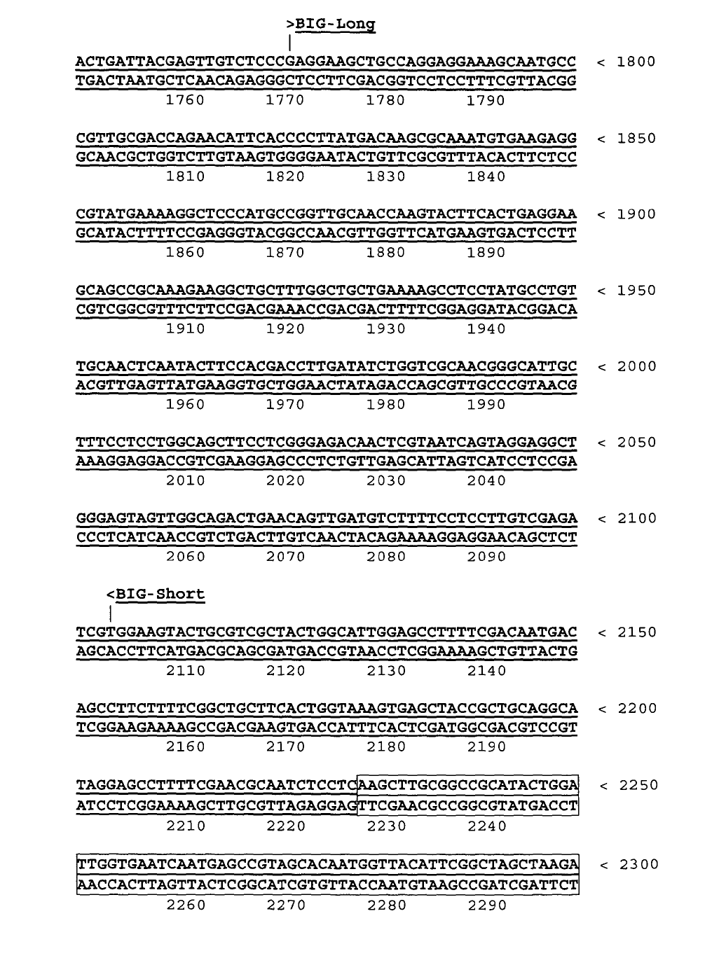

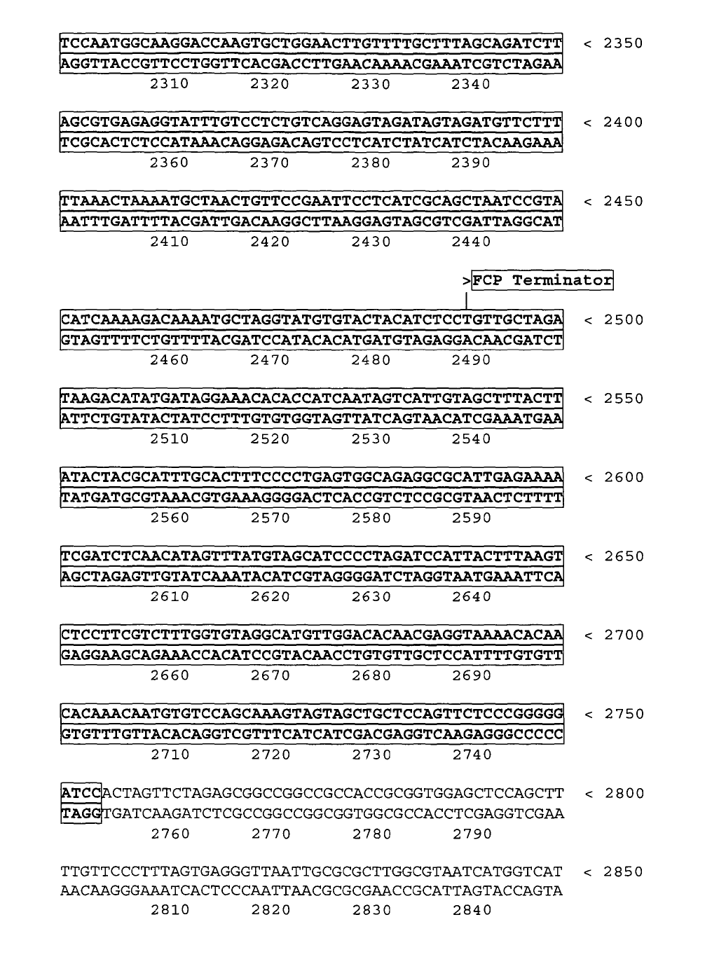

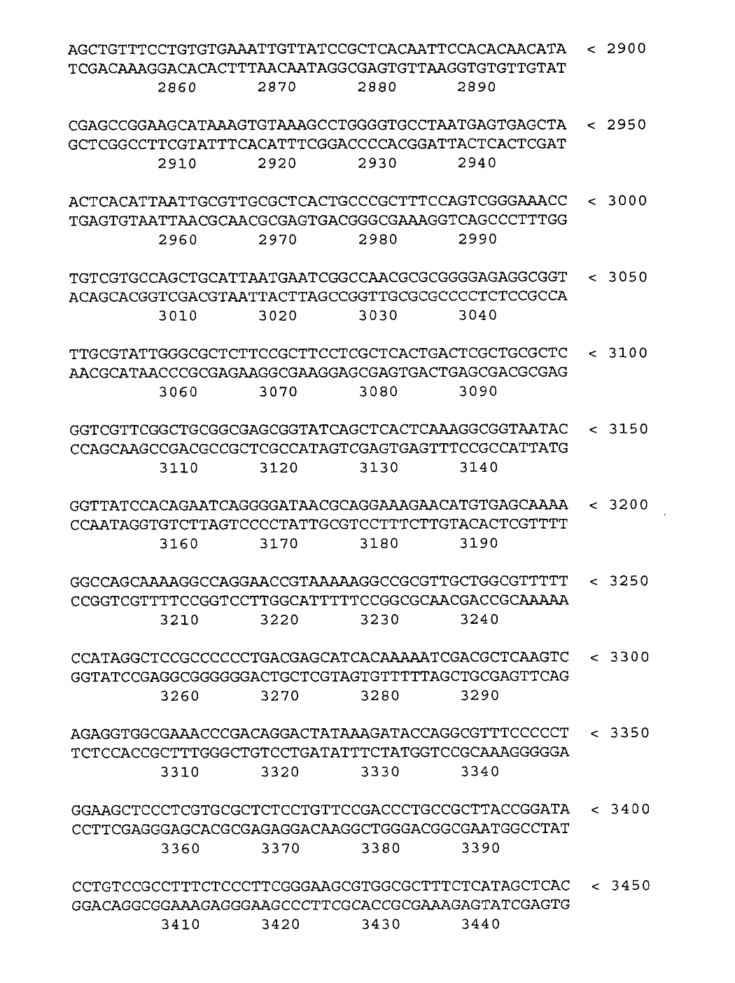

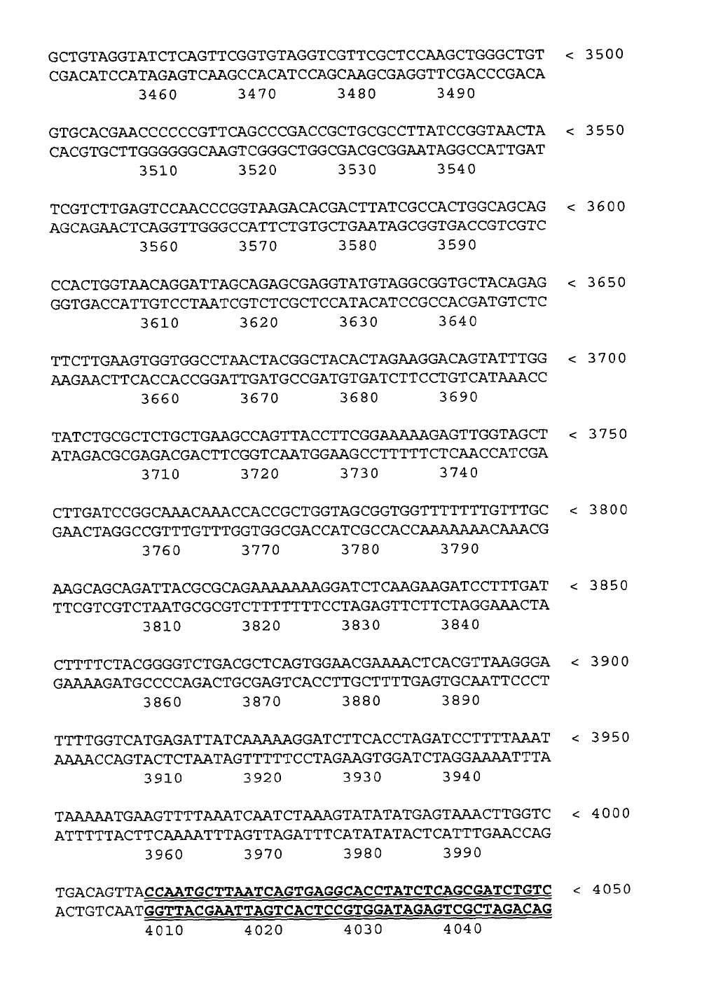

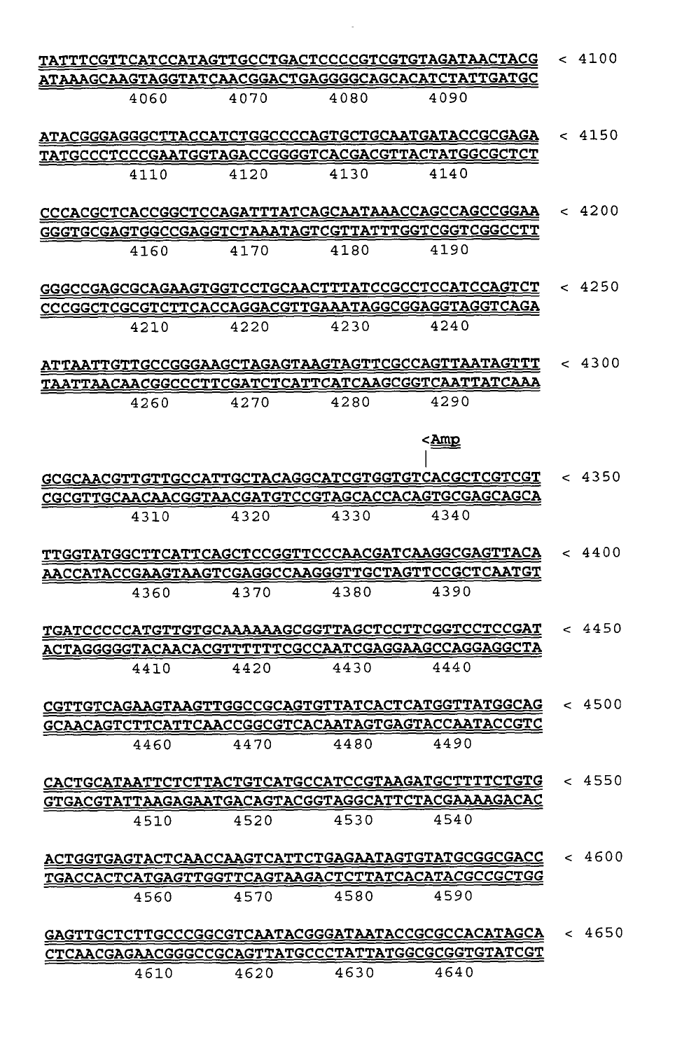

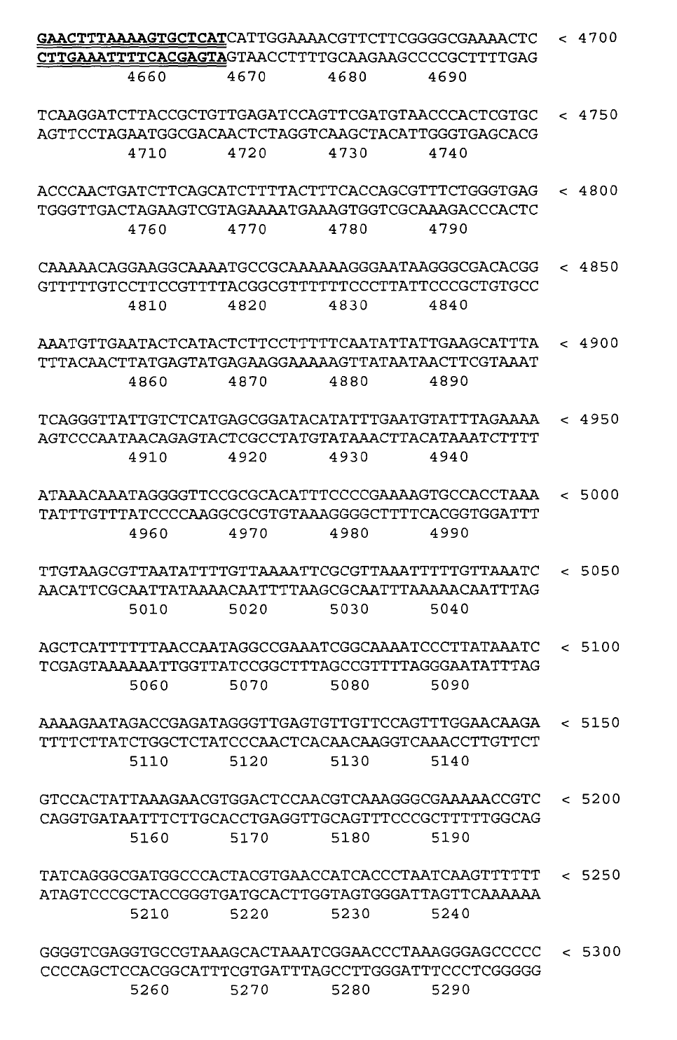

To confirm the role of the BIG1 gene product, the gene was knocked down using RNA interference (RNAi). This was achieved using the same expression cassette as that used for construction of an over expression vector (Poulsen et al. 2006) in addition to a second cassette reported in the same work containing an FCP promotor for constitutive expression. Primers were designed to amplify bases 33-282 of the cDNA of BIG1 and introduce restriction sites to allow the fragment to be inserted into the cassette in the antisense direction. This resulted in a vector producing a strand of antisense RNA that interacts with the cellular BIG1 messenger RNA activating poorly understood silencing mechanisms within the cell. A second silencing strategy employed a primer pair to amplify a longer fragment (bases 33-446) of the BIG1 cDNA. These primers also introduced restriction enzyme sites, allowing both the fragments to be inserted into the cassette in an inverted repeat, the resulting double stranded RNA also activates gene silencing mechanisms. The vectors produced are shown in FIGS. 12 to 15. Wildtype Thalassiosira pseudonana was transformed using the Biorad Biolistics particle delivery system.

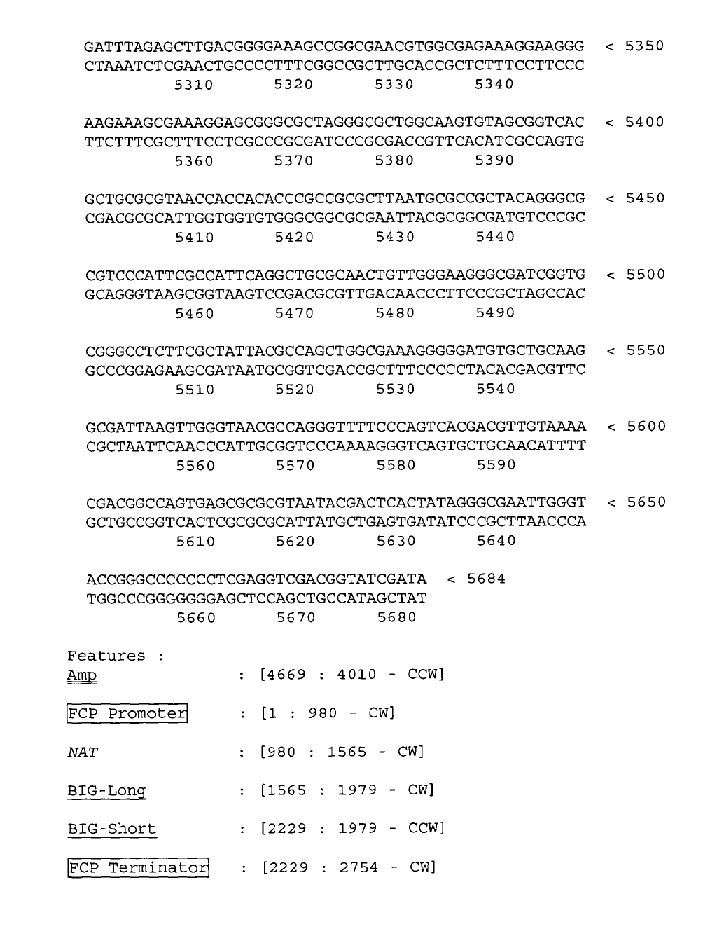

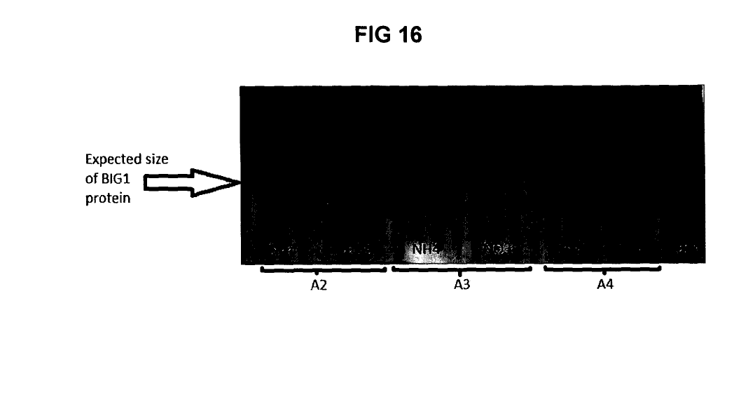

Transformants were screened by Western blot targeting the BIG1 protein using a 1:1000 dilution of an antipeptide serum (shown in FIG. 16). To achieve this proteins were extracted from 50 ml of culture from the 6^{th} day of stationary phase of growth (determined to be the phase when the greatest concentration of the BIG1 protein was present in wild type cells) by pelleting the cells by centrifuging at 4,000 rpm at 4° C. for 10 mins in a bench-top centrifuge, the supernatant discarded and the pellet resuspended in 50 μl protein lysis buffer (50 mM Tris pH 6.8, 2% SDS) and incubated at room temperature for 30 min before centrifuging at 13,000 rpm at 4° C. for 10 mins. The protein-containing supernatant was taken off and pelleted cell debris discarded. The concentration of the retained protein was determined using the BCA (bicinchoninic acid) quantification kit (Pierce, Thermo Scientific). 30 μg of protein samples were denatured with laemmli buffer at 95° C. for 10 min before loading on a 10% polyacrylamide gel (10% polyacrylamide, 0.375 M Tris HCl pH 8.8, 0.1% SDS, 6.25×10⁻⁴% w/v APS, 1/800 volume TEMED). The proteins were separated off the gels by electrophoresis at 100 V for 2.5 h in 1× Tris-glycine running buffer (10×: Tris base 30.3 g L-1, glycine 144 g L-1, SDS 10 g L-1) then transferred onto nitrocellulose “protran” membrane (Schleicher and Shuell) using the Criterion blotter system (Biorad) at 100 V for 1 h. Protein transfer and loading quantities were checked using the reversible protein stain Ponceau S by incubating the membranes with Ponceau S solution (0.1%(w/v) Ponceau S in 5%(v/v) acetic acid) for 5 minutes at room temperature with gentle agitation, followed by 3 rinses with MilliQ water. Membranes were then blocked for 1 h in 5% non-fat dry milk powder dissolved in PBST (1×PBS, 0.01% Tween 20), then hybridised with the BIG1 antiserum diluted 1:1,000 in PBST at 4° C. overnight (or at room temperature for 4 h) all under gentle agitation. The membranes were then washed 3 times in PBST with gentle agitation for 10 min before hybridising with 1:10,000 anti-rabbit IgG HRP (horseradish peroxidase) conjugate secondary antibody (Promega) diluted in 5% milk PBST for 1 h at room temperature with gentle agitation. The membranes were washed a further three times with PBST with gentle agitation for 10 min before being incubated with ECL (enhanced chemiluminescent) substrate (Pierce, Thermo Scientific) for 2 min at room temperature to detect the activity of the secondary antibody and the image captured using a CCD camera (Fuji LASimager 3000).

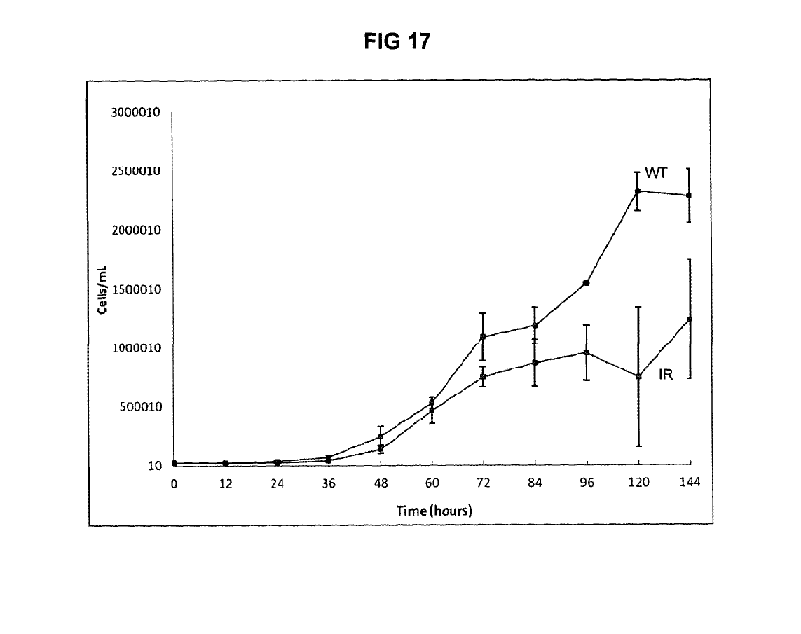

The phenotype of a knockdown clone transformed with inverted repeat construct was assessed through a growth experiment comparing its growth with that of wildtype cells (FIG. 17). Cells were grown in nitrate limited NEPCC (http://www3.botany.ubc.ca/cccm/NEPCC/esaw.html) containing three times the usual concentration of nutrient stocks but only 100 μM concentrations of NaNO₃ (which is 549 μM in replete NEPCC) until the 6^{th} day after entering the stationary phase, identified as the first day that the fv/fm falls below 0.6, when 25000 cells ml⁻¹ were inoculated into 20 ml replete NEPCC and the cells were counted with a multisizer coulter counter (Beckman) every 12 hours.

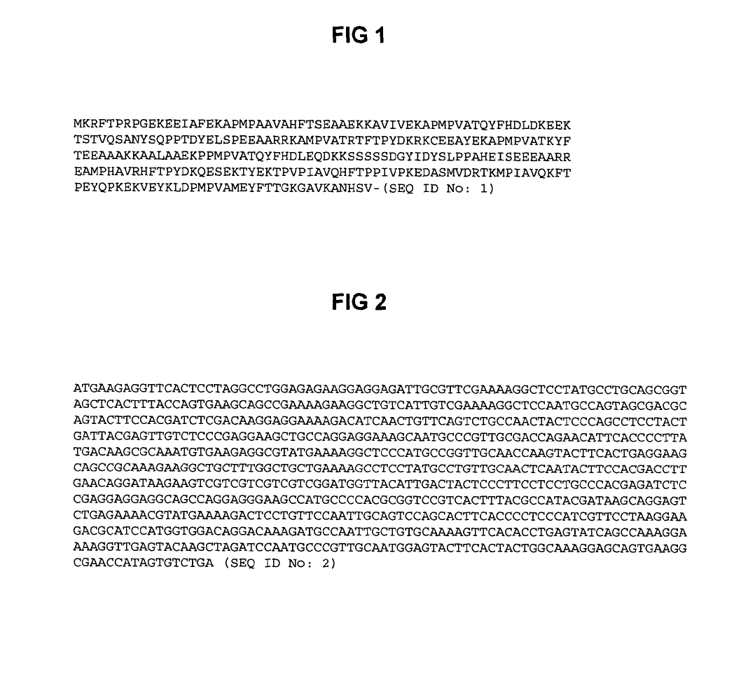

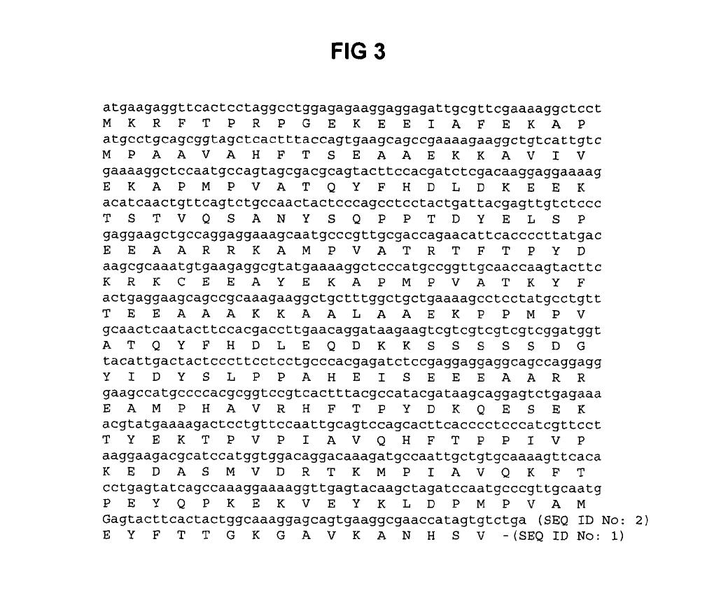

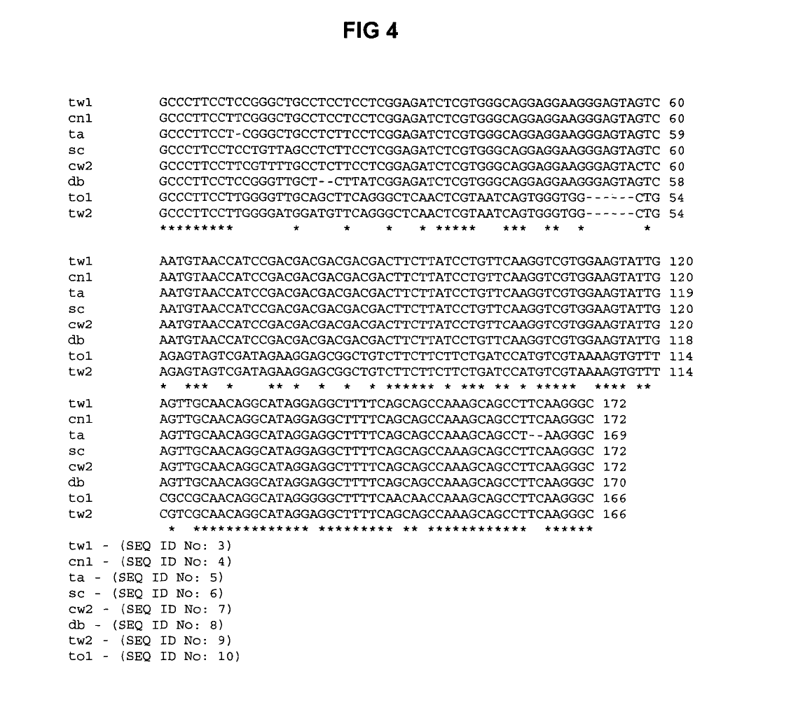

1. A nucleic acid molecule encoding a polypeptide capable of enhancing the rate of cell-division of a microorganism or plant cell wherein said polypeptide comprises an amino acid sequence having at least 75% amino acid sequence identity over the entire length of the amino acid sequence of FIG. 1 (SEQ ID No: 1) or a nucleic acid molecule complementary thereto.

2. A nucleic acid molecule as claimed in claim 1 which encodes a polypeptide comprising the amino acid sequence set forth in FIG. 1 (SEQ ID No: 1).

3. A nucleic acid molecule encoding a polypeptide capable of enhancing the rate of cell-division of a microorganism or a plant cell wherein said nucleic acid molecule comprises a nucleotide sequence having at least 75% identity over the entire length of the nucleotide sequence of FIG. 2 (SEQ ID No: 2).

4. A nucleic acid molecule as claimed in claim 3 comprising the sequence of nucleotides set forth in FIG. 2 (SEQ ID No: 2).

5. An expression vector comprising a nucleic acid molecule as claimed in claim 1.

6. A host cell transformed or transfected with a vector as claimed in claim 5, optionally wherein the host cell is selected from a yeast, a fungal cell, an algal cell, a diatom, or a plant cell.

7.-8. (canceled)

9. A host cell of claim 6 wherein said cell is a photosynthetic cell.

10. A plant comprising a cell as claimed in claim 6.

11. An algal culture comprising a cell as claimed in claim 6.

12. A vector comprising the antisense of a nucleic acid molecule as claimed in claim 1, or a fragment thereof, under the control of a promoter, optionally wherein the fragment is nucleotides 33 to 282 of the nucleotide sequence set forth in FIG. 2 (SEQ ID NO:2).

13. (canceled)

14. A vector comprising an inverted repeat of a nucleic acid molecule as claimed in claim 1, or a fragment thereof, under the control of a promoter, optionally wherein the fragment is nucleotides 33 to 446 of the nucleotide sequence set forth in FIG. 2 (SEQ ID NO:2).

15. (canceled)

16. A polypeptide capable of enhancing the rate of cell-division of a microorganism or plant cell wherein said polypeptide comprises an amino acid sequence having at least 75% amino acid sequence identity over the entire length of the amino acid sequence set forth in FIG. 1 (SEQ ID No: 1) optionally wherein the polypeptide comprises the amino acid sequence set forth in FIG. 1 (SEQ ID No: 1).

17. (canceled)

18. A method for enhancing the rate of cell-division of a microorganism or plant cell comprising transforming or transfecting said microorganism or plant cell with a nucleic acid molecule encoding a polypeptide capable of enhancing the rate of cell-division of a microorganism or plant cell wherein said polypeptide comprises an amino acid sequence having at least 50% amino acid sequence similarity with amino acids 128 to 184 of the amino acid sequence set forth in FIG. 1 (SEQ ID No: 1) or a nucleic acid molecule complementary thereto such that the polypeptide encoded by said nucleic acid is expressed therein.

19. The method as claimed in claim 18, wherein said polypeptide comprises
(a) an amino acid sequence having at least 50% amino acid sequence identity with amino acids 128 to 184 of the amino acid sequence set forth in FIG. 1 (SEQ ID No: 1) or a nucleic acid molecule complementary thereto,
(b) an amino acid sequence having at least 50% amino acid sequence similarity with the amino acid sequence set forth in FIG. 1 (SEQ ID No: 1) or a nucleic acid molecule complementary thereto, or
(c) an amino acid sequence having at least 50% amino acid sequence identity with the amino acid sequence set forth in FIG. 1 (SEQ ID No: 1) or a nucleic acid molecule complementary thereto.

20.-21. (canceled)

22. A method for enhancing the rate of cell-division of a microorganism or plant cell comprising transforming or transfecting said microorganism or plant cell with a nucleic acid molecule as claim in claim 1 such that the polypeptide encoded by said nucleic acid is expressed therein.

23. A method for enhancing the rate of cell-division of a microorganism or plant cell comprising contacting said microorganism or plant cell with a polypeptide capable of enhancing the rate of cell-division of a microorganism or plant cell wherein said polypeptide comprises an amino acid sequence having at least 50% amino acid similarity with amino acids 128 to 184 of the amino acid sequence set forth in FIG. 1 (SEQ ID No: 1).

24. The method of claim 23, wherein said polypeptide
(a) has an amino acid sequence identity of at least 50% with amino acids 128 to 184 of the amino acid sequence set forth in FIG. 1 (SEQ ID No: 1),
(b) comprises an amino acid sequence having at least 50% amino acid similarity with the amino acid sequence set forth in FIG. 1 (SEQ ID No: 1),
(c) has an amino acid sequence identity of at least 50% with the amino acid sequence set forth in FIG. 1 (SEQ ID No: 1),
(d) has an amino acid sequence identity of at least 50% over the entire length of the amino acid sequence set forth in FIG. 1 (SEQ ID No: 1), or
(e) comprises the amino acid sequence set forth in FIG. 1 (SEQ ID No: 1).

25.-28. (canceled)

29. A method as claimed in claim 18 wherein said microorganism is a yeast, a fungal cell, an algal cell or a plant cell, optionally where said microorganism is an algae.

30. (canceled)

31. The method of claim 29 wherein said algae is a diatom.

32. A method as claimed in claim 18 wherein said microorganism or plant cell
(a) produces a biofuel, or
(b) produces one or more long-chain polyunsaturated fatty acids.

33. (canceled)

34. A microorganism or plant cell produced by the method as claimed in claim 18.

35. A plant cultivated from the plant cell of claim 34.

36. A composition comprising the polypeptide of claim 16 or 17.

37. Use of a microorganism or plant cell of claim 34 in any one of the processes set forth in Tables 1 or 2 or to produce one or more of the products set forth in Tables 1 or 2.

38. The use as claimed in claim 37 wherein said microorganism is an algae.

39. A microorganism which is, or has the identifying characteristics of, a strain of Thalassiosira pseudonana deposited with the Culture Collection of Algae and Protozoa under the accession number CCAP 1085/23, or a mutant strain derived therefrom.
